SEQUENCE LISTING (7 sequences)

SEQ ID NO: 1 - DNA, 5292 bp, Homo sapiens
  misc_feature (1)..(254): exon
  misc_feature (188)..(2770): LDLR isoform 1 encoded by full-length CDS, 188-2770;  other variants encoded by alternative splice variants missing an exon; most common variant missing fourth exon or twelfth exon
  misc_signal (188)..(250)
  misc_feature (251)..(2767): Mature protein of isoform 1
  misc_feature (255)..(377): exon
  misc_feature (378)..(500): exon
  misc_feature (501)..(881): exon
  misc_feature (882)..(1004): exon
  misc_feature (1005)..(1127): exon
  misc_feature (1128)..(1247): exon
  misc_feature (1248)..(1373): exon
  misc_feature (1374)..(1545): exon
  misc_feature (1546)..(1773): exon
  misc_feature (1774)..(1892): exon
  misc_feature (1893)..(2032): exon
  misc_feature (2033)..(2174): exon
  misc_feature (2175)..(2327): exon
  misc_feature (2328)..(2498): exon
  polyA_signal (5252)..(5257)
  polyA_site (5284)..(5284)
ctcttgcagtgaggtgaagacatttgaaaatcaccccactgcaaactcctccccctgctagaaacctcacattgaaatgctgtaaatgacgtgggccccgagtgcaatcgcgggaagccagggtttccagctaggacacagcaggtcgtgatccgggtcgggacactgcctggcagaggctgcgagcatggggccctggggctggaaattgcgctggaccgtcgccttgctcctcgccgcggcggggactgcagtgggcgacagatgcgaaagaaacgagttccagtgccaagacgggaaatgcatctcctacaagtgggtctgcgatggcagcgctgagtgccaggatggctctgatgagtcccaggagacgtgcttgtctgtcacctgcaaatccggggacttcagctgtgggggccgtgtcaaccgctgcattcctcagttctggaggtgcgatggccaagtggactgcgacaacggctcagacgagcaaggctgtccccccaagacgtgctcccaggacgagtttcgctgccacgatgggaagtgcatctctcggcagttcgtctgtgactcagaccgggactgcttggacggctcagacgaggcctcctgcccggtgctcacctgtggtcccgccagcttccagtgcaacagctccacctgcatcccccagctgtgggcctgcgacaacgaccccgactgcgaagatggctcggatgagtggccgcagcgctgtaggggtctttacgtgttccaaggggacagtagcccctgctcggccttcgagttccactgcctaagtggcgagtgcatccactccagctggcgctgtgatggtggccccgactgcaaggacaaatctgacgaggaaaactgcgctgtggccacctgtcgccctgacgaattccagtgctctgatggaaactgcatccatggcagccggcagtgtgaccgggaatatgactgcaaggacatgagcgatgaagttggctgcgttaatgtgacactctgcgagggacccaacaagttcaagtgtcacagcggcgaatgcatcaccctggacaaagtctgcaacatggctagagactgccgggactggtcagatgaacccatcaaagagtgcgggaccaacgaatgcttggacaacaacggcggctgttcccacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctccatcgcctacctcttcttcaccaaccggcacgaggtcaggaagatgacgctggaccggagcgagtacaccagcctcatccccaacctgaggaacgtggtcgctctggacacggaggtggccagcaatagaatctactggtctgacctgtcccagagaatgatctgcagcacccagcttgacagagcccacggcgtctcttcctatgacaccgtcatcagcagagacatccaggcccccgacgggctggctgtggactggatccacagcaacatctactggaccgactctgtcctgggcactgtctctgttgcggataccaagggcgtgaagaggaaaacgttattcagggagaacggctccaagccaagggccatcgtggtggatcctgttcatggcttcatgtactggactgactggggaactcccgccaagatcaagaaagggggcctgaatggtgtggacatctactcgctggtgactgaaaacattcagtggcccaatggcatcaccctagatctcctcagtggccgcctctactgggttgactccaaacttcactccatctcaagcatcgatgtcaacgggggcaaccggaagaccatcttggaggatgaaaagaggctggcccaccccttctccttggccgtctttgaggacaaagtattttggacagatatcatcaacgaagccattttcagtgccaaccgcctcacaggttccgatgtcaacttgttggctgaaaacctactgtccccagaggatatggttctcttccacaacctcacccagccaagaggagtgaactggtgtgagaggaccaccctgagcaatggcggctgccagtatctgtgcctccctgccccgcagatcaacccccactcgcccaagtttacctgcgcctgcccggacggcatgctgctggccagggacatgaggagctgcctcacagaggctgaggctgcagtggccacccaggagacatccaccgtcaggctaaaggtcagctccacagccgtaaggacacagcacacaaccacccgacctgttcccgacacctcccggctgcctggggccacccctgggctcaccacggtggagatagtgacaatgtctcaccaagctctgggcgacgttgctggcagaggaaatgagaagaagcccagtagcgtgagggctctgtccattgtcctccccatcgtgctcctcgtcttcctttgcctgggggtcttccttctatggaagaactggcggcttaagaacatcaacagcatcaactttgacaaccccgtctatcagaagaccacagaggatgaggtccacatttgccacaaccaggacggctacagctacccctcgagacagatggtcagtctggaggatgacgtggcgtgaacatctgcctggagtcccgtccctgcccagaacccttcctgagacctcgccggccttgttttattcaaagacagagaagaccaaagcattgcctgccagagctttgttttatatatttattcatctgggaggcagaacaggcttcggacagtgcccatgcaatggcttgggttgggattttggtttcttcctttcctcgtgaaggataagagaaacaggcccggggggaccaggatgacacctccatttctctccaggaagttttgagtttctctccaccgtgacacaatcctcaaacatggaagatgaaaggggaggggatgtcaggcccagagaagcaagtggctttcaacacacaacagcagatggcaccaacgggaccccctggccctgcctcatccaccaatctctaagccaaacccctaaactcaggagtcaacgtgtttacctcttctatgcaagccttgctagacagccaggttagcctttgccctgtcacccccgaatcatgacccacccagtgtctttcgaggtgggtttgtaccttccttaagccaggaaagggattcatggcgtcggaaatgatctggctgaatccgtggtggcaccgagaccaaactcattcaccaaatgatgccacttcccagaggcagagcctgagtcactggtcacccttaatatttattaagtgcctgagacacccggttaccttggccgtgaggacacgtggcctgcacccaggtgtggctgtcaggacaccagcctggtgcccatcctcccgacccctacccacttccattcccgtggtctccttgcactttctcagttcagagttgtacactgtgtacatttggcatttgtgttattattttgcactgttttctgtcgtgtgtgttgggatgggatcccaggccagggaaagcccgtgtcaatgaatgccggggacagagaggggcaggttgaccgggacttcaaagccgtgatcgtgaatatcgagaactgccattgtcgtctttatgtccgcccacctagtgcttccacttctatgcaaatgcctccaagccattcacttccccaatcttgtcgttgatgggtatgtgtttaaaacatgcacggtgaggccgggcgcagtggctcacgcctgtaatcccagcactttgggaggccgaggcgggtggatcatgaggtcaggagatcgagaccatcctggctaacacgtgaaaccccgtctctactaaaaatacaaaaaattagccgggcgtggtggcgggcacctgtagtcccagctactcgggaggctgaggcaggagaatggtgtgaacccgggaagcggagcttgcagtgagccgagattgcgccactgcagtccgcagtctggcctgggcgacagagcgagactccgtctcaaaaaaaaaaaacaaaaaaaaaccatgcatggtgcatcagcagcccatggcctctggccaggcatggcgaggctgaggtgggaggatggtttgagctcaggcatttgaggctgtcgtgagctatgattatgccactgctttccagcctgggcaacatagtaagaccccatctcttaaaaaatgaatttggccagacacaggtgcctcacgcctgtaatcccagcactttgggaggctgagctggatcacttgagttcaggagttggagaccaggcctgagcaacaaagcgagatcccatctctacaaaaaccaaaaagttaaaaatcagctgggtacggtggcacgtgcctgtgatcccagctacttgggaggctgaggcaggaggatcgcctgagcccaggaggtggaggttgcagtgagccatgatcgagccactgcactccagcctgggcaacagatgaagaccctatttcagaaatacaactataaaaaaataaataaatcctccagtctggatcgtttgacgggacttcaggttctttctgaaatcgccgtgttactgttgcactgatgtccggagagacagtgacagcctccgtcagactcccgcgtgaagatgtcacaagggattggcaattgtccccagggacaaaacactgtgtcccccccagtgcagggaaccgtgataagcctttctggtttcggagcacgtaaatgcgtccctgtacagatagtggggattttttgttatgtttgcactttgtatattggttgaaactgttatcacttatatatatatatatacacacatatatataaaatctatttatttttgcaaaccctggttgctgtatttgttcagtgactattctcggggccctgtgtagggggttattgcctctgaaatgcctcttctttatgtacaaagattatttgcacgaactggactgtgtgcaacgctttttgggagaatgatgtccccgttgtatgtatgagtggcttctgggagatgggtgtcactttttaaaccactgtatagaaggtttttgtagcctgaatgtcttactgtgatcaattaaatttcttaaatgaaccaatttgtctaaaaaaaaaaa

SEQ ID NO: 2 - DNA, 2583 bp, Homo sapiens
  CDS (1)..(2583)
atggggccctggggctggaaattgcgctggaccgtcgccttgctcctcgccgcggcggggactgcagtgggcgacagatgtgaaagaaacgagttccagtgccaagacgggaaatgcatctcctacaagtgggtctgcgatggcagcgctgagtgccaggatggctctgatgagtcccaggagacgtgcttgtctgtcacctgcaaatccggggacttcagctgtgggggccgtgtcaaccgctgcattcctcagttctggaggtgcgatggccaagtggactgcgacaacggctcagacgagcaaggctgtccccccaagacgtgctcccaggacgagtttcgctgccacgatgggaagtgcatctctcggcagttcgtctgtgactcagaccgggactgcttggacggctcagacgaggcctcctgcccggtgctcacctgtggtcccgccagcttccagtgcaacagctccacctgcatcccccagctgtgggcctgcgacaacgaccccgactgcgaagatggctcggatgagtggccgcagcgctgtaggggtctttacgtgttccaaggggacagtagcccctgctcggccttcgagttccactgcctaagtggcgagtgcatccactccagctggcgctgtgatggtggccccgactgcaaggacaaatctgacgaggaaaactgcgctgtggccacctgtcgccctgacgaattccagtgctctgatggaaactgcatccatggcagccggcagtgtgaccgggaatatgactgcaaggacatgagcgatgaagttggctgcgttaatgtgacactctgcgagggacccaacaagttcaagtgtcacagcggcgaatgcatcaccctggacaaagtctgcaacatggctagagactgccgggactggtcagatgaacccatcaaagagtgcgggaccaacgaatgcttggacaacaacggcggctgttcccacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctccatcgcctacctcttcttcaccaaccggcacgaggtcaggaagatgacgctggaccggagcgagtacaccagcctcatccccaacctgaggaacgtggtcgctctggacacggaggtggccagcaatagaatctactggtctgacctgtcccagagaatgatctgcagcacccagcttgacagagcccacggcgtctcttcctatgacaccgtcatcagcagggacatccaggcccccgacgggctggctgtggactggatccacagcaacatctactggaccgactctgtcctgggcactgtctctgttgcggataccaagggcgtgaagaggaaaacgttattcagggagaacggctccaagccaagggccatcgtggtggatcctgttcatggcttcatgtactggactgactggggaactcccgccaagatcaagaaagggggcctgaatggtgtggacatctactcgctggtgactgaaaacattcagtggcccaatggcatcaccctagatctcctcagtggccgcctctactgggttgactccaaacttcactccatctcaagcatcgatgtcaatgggggcaaccggaagaccatcttggaggatgaaaagaggctggcccaccccttctccttggccgtctttgaggacaaagtattttggacagatatcatcaacgaagccattttcagtgccaaccgcctcacaggttccgatgtcaacttgttggctgaaaacctactgtccccagaggatatggtcctcttccacaacctcacccagccaagaggagtgaactggtgtgagaggaccaccctgagcaatggcggctgccagtatctgtgcctccctgccccgcagatcaacccccactcgcccaagtttacctgcgcctgcccggacggcatgctgctggccagggacatgaggagctgcctcacagaggctgaggctgcagtggccacccaggagacatccaccgtcaggctaaaggtcagctccacagccgtaaggacacagcacacaaccacccggcctgttcccgacacctcccggctgcctggggccacccctgggctcaccacggtggagatagtgacaatgtctcaccaagctctgggcgacgttgctggcagaggaaatgagaagaagcccagtagcgtgagggctctgtccattgtcctccccatcgtgctcctcgtcttcctttgcctgggggtcttccttctatggaagaactggcggcttaagaacatcaacagcatcaactttgacaaccccgtctatcagaagaccacagaggatgaggtccacatttgccacaaccaggacggctacagctacccctcgagacagatggtcagtctggaggatgacgtggcgtag

SEQ ID NO: 3 - protein, 860 aa, Homo sapiens [sequence omitted]

SEQ ID NO: 4 - DNA, 2583 bp, Artificial Sequence
  Artificial hLDLR
  misc_feature (1)..(2583): Artificial hLDLR coding sequence
atgggaccttggggttggaaactccgctggacagtggctctgctcctggcagcagcaggaacagccgtgggagatcgctgcgaaaggaacgagttccagtgccaggacggcaagtgcatcagctacaagtgggtctgcgacggtagcgcagagtgtcaggacggaagcgacgaaagccaggagacttgcctgagcgtgacttgcaagtccggcgacttctcttgcggaggcagagtgaaccgctgcatccctcagttttggcggtgcgacggccaggtggattgcgataacggaagcgacgagcagggttgccctcctaagacttgcagccaggacgaattccgctgtcacgacggcaagtgcatcagcaggcagttcgtctgcgacagcgacagggattgtctggacggaagcgacgaggcctcttgtcctgtgctgacttgtggcccagccagcttccagtgcaactccagcacttgcatcccacagctctgggcttgcgacaacgacccagattgcgaggacggatcagacgagtggccacagcgctgcagaggcctgtacgtgtttcagggcgattccagcccttgcagcgcttttgagttccactgcctgagcggcgagtgcattcactcttcttggaggtgcgacggtggcccagattgcaaggacaagagcgacgaggagaattgcgccgtggctacttgcagaccagacgaattccagtgcagcgacggcaattgcatccacggctctaggcagtgcgacagggagtacgattgcaaggacatgagcgacgaagtcggttgcgtgaacgtcaccctctgcgagggtcccaataagttcaagtgccacagcggcgagtgcattaccctggacaaggtctgcaacatggccagggattgccgggattggagcgacgagcctatcaaggagtgcggcaccaacgagtgcctggataacaacggcggctgcagccacgtgtgcaatgacctgaagatcggctacgagtgcctctgcccagacggattccagctggtggctcagagacgctgcgaagacatcgacgagtgccaggatcccgacacttgcagccagctgtgcgtgaatctggagggaggctacaagtgccagtgcgaagagggattccagctggaccctcacaccaaggcttgtaaagccgtgggcagcatcgcctacctgttcttcaccaacagacacgaagtgcggaagatgaccctggatcggagcgagtacaccagcctgatccctaacctgcggaacgtggtggccctggatacagaagtggccagcaacaggatctattggagcgacctgagccagcggatgatttgcagcacccagctggacagagcccacggagtgtccagctacgacaccgtgatcagcagagacatccaggctccagacggactggcagtggattggatccacagcaacatctactggaccgactcagtgctgggaacagtgtccgtggccgatacaaagggcgtgaagcggaagaccctgttcagagagaacggcagcaagcccagggctattgtggtggatcccgtgcacggcttcatgtattggaccgattggggcacccccgctaagatcaagaagggcggcctgaacggcgtggacatctacagcctggtgaccgagaacatccagtggcccaacggaattaccctggacctgctgagcggcagactgtattgggtggacagcaagctgcacagcatcagcagcatcgacgtgaacggcggaaaccggaagaccatcctggaggacgagaagagactggcccaccctttcagcctggccgtgttcgaggacaaggtcttctggaccgacatcatcaacgaggccatcttcagcgccaacaggctgacaggaagcgacgtgaacctgctggcagagaatctgctgtctcccgaggacatggtgctgttccacaacctgacccagcccagaggcgtcaattggtgcgagagaaccaccctgagcaacggaggttgccagtacctgtgcctgccagcccctcagattaaccctcacagccccaagttcacttgcgcttgcccagacggcatgctgctggccagagatatgcggtcttgtctgacagaagccgaagccgctgtggctacacaggagacaagcaccgtgcggctgaaggtgtctagcacagccgtgagaacccagcacacaaccaccagacccgtgccagataccagcagactgccaggagctacaccaggactgaccaccgtggagatcgtgaccatgagccaccaggctctgggagacgtggcaggaagaggaaacgagaagaagcctagcagcgtgagagccctgtctatcgtgctgcccatcgtgctgctggtgttcctctgtctgggcgtgttcctcctctggaagaattggcggctgaagaacatcaacagcatcaacttcgacaaccccgtgtaccagaagaccaccgaggacgaggtgcacatttgccacaaccaggacggctacagctaccctagcaggcagatggtgtccctggaagacgacgtggcttga

SEQ ID NO: 5 - protein, 738 aa, Artificial Sequence
  Adeno-associated virus 8 vp1 capsid protein
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 6 - DNA, 6768 bp, Artificial Sequence
  pAAV.TBG.PI.hLDLRco.RGB
  repeat_region (1)..(130): 5′ ITR
  enhancer (221)..(320): Alpha mic/bik
  enhancer (327)..(426): Alpha mic/bik
  promoter (442)..(901): TBG
  TATA_signal (885)..(888): TATA
  CDS (969)..(3551): codon optimized hLDLR
  polyA_signal (3603)..(3729): Rabbit globin poly A
  repeat_region (3818)..(3947): 3′ ITR
  rep_origin (4124)..(4579): f1 ori
  misc_feature (4710)..(5567): AP(R)
  rep_origin (5741)..(6329): Origin of replication
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagaattcgcccttaagctagcaggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagacagctttattgcggtagtttatcacagttaaattgctaacgcagacgttgcatgggaccttggggttggaaactccgctggacagtggctctgctcctggcagcagcaggaacagccgtgggagatcgctgcgaaaggaacgagttccagtgccaggacggcaagtgcatcagctacaagtgggtctgcgacggtagcgcagagtgtcaggacggaagcgacgaaagccaggagacttgcctgagcgtgacttgcaagtccggcgacttctcttgcggaggcagagtgaaccgctgcatccctcagttttggcggtgcgacggccaggtggattgcgataacggaagcgacgagcagggttgccctcctaagacttgcagccaggacgaattccgctgtcacgacggcaagtgcatcagcaggcagttcgtctgcgacagcgacagggattgtctggacggaagcgacgaggcctcttgtcctgtgctgacttgtggcccagccagcttccagtgcaactccagcacttgcatcccacagctctgggcttgcgacaacgacccagattgcgaggacggatcagacgagtggccacagcgctgcagaggcctgtacgtgtttcagggcgattccagcccttgcagcgcttttgagttccactgcctgagcggcgagtgcattcactcttcttggaggtgcgacggtggcccagattgcaaggacaagagcgacgaggagaattgcgccgtggctacttgcagaccagacgaattccagtgcagcgacggcaattgcatccacggctctaggcagtgcgacagggagtacgattgcaaggacatgagcgacgaagtcggttgcgtgaacgtcaccctctgcgagggtcccaataagttcaagtgccacagcggcgagtgcattaccctggacaaggtctgcaacatggccagggattgccgggattggagcgacgagcctatcaaggagtgcggcaccaacgagtgcctggataacaacggcggctgcagccacgtgtgcaatgacctgaagatcggctacgagtgcctctgcccagacggattccagctggtggctcagagacgctgcgaagacatcgacgagtgccaggatcccgacacttgcagccagctgtgcgtgaatctggagggaggctacaagtgccagtgcgaagagggattccagctggaccctcacaccaaggcttgtaaagccgtgggcagcatcgcctacctgttcttcaccaacagacacgaagtgcggaagatgaccctggatcggagcgagtacaccagcctgatccctaacctgcggaacgtggtggccctggatacagaagtggccagcaacaggatctattggagcgacctgagccagcggatgatttgcagcacccagctggacagagcccacggagtgtccagctacgacaccgtgatcagcagagacatccaggctccagacggactggcagtggattggatccacagcaacatctactggaccgactcagtgctgggaacagtgtccgtggccgatacaaagggcgtgaagcggaagaccctgttcagagagaacggcagcaagcccagggctattgtggtggatcccgtgcacggcttcatgtattggaccgattggggcacccccgctaagatcaagaagggcggcctgaacggcgtggacatctacagcctggtgaccgagaacatccagtggcccaacggaattaccctggacctgctgagcggcagactgtattgggtggacagcaagctgcacagcatcagcagcatcgacgtgaacggcggaaaccggaagaccatcctggaggacgagaagagactggcccaccctttcagcctggccgtgttcgaggacaaggtcttctggaccgacatcatcaacgaggccatcttcagcgccaacaggctgacaggaagcgacgtgaacctgctggcagagaatctgctgtctcccgaggacatggtgctgttccacaacctgacccagcccagaggcgtcaattggtgcgagagaaccaccctgagcaacggaggttgccagtacctgtgcctgccagcccctcagattaaccctcacagccccaagttcacttgcgcttgcccagacggcatgctgctggccagagatatgcggtcttgtctgacagaagccgaagccgctgtggctacacaggagacaagcaccgtgcggctgaaggtgtctagcacagccgtgagaacccagcacacaaccaccagacccgtgccagataccagcagactgccaggagctacaccaggactgaccaccgtggagatcgtgaccatgagccaccaggctctgggagacgtggcaggaagaggaaacgagaagaagcctagcagcgtgagagccctgtctatcgtgctgcccatcgtgctgctggtgttcctctgtctgggcgtgttcctcctctggaagaattggcggctgaagaacatcaacagcatcaacttcgacaaccccgtgtaccagaagaccaccgaggacgaggtgcacatttgccacaaccaggacggctacagctaccctagcaggcagatggtgtccctggaagacgacgtggcttgataagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 7 - protein, 860 aa, Artificial Sequence [sequence omitted]

(19) United States
(12) Patent Application Publication
(10) Pub. No.: US 2022/0040332 A1
(43) Pub. Date: Feb. 10, 2022

(54) GENE THERAPY FOR TREATING FAMILIAL HYPERCHOLESTEROLEMIA

(71) Applicant: The Trustees of the University of Pennsylvania, Philadelphia, PA (US)

(72) Inventor: Daniel J. Rader, Philadelphia, PA (US)

(21) Appl. No.: 17/414,263

(22) PCT Filed: Dec. 19, 2019
(86) PCT No.: PCT/US2019/067316

§ 371 Date: Jun. 15, 2021

Related U.S. Application Data
(60) Provisional application No. 62/782,627, filed on Dec. 20, 2018.

(51) Int. Cl. A61K 48/00; A61K 31/573; A61P 3/06

(52) U.S. Cl. CPC A61K 48/0058; C12N 2750/14143; A61P 3/06; A61K 31/573

(57) ABSTRACT

Regimens useful treating a human patient having familial hypercholesterolemia are described. Such regimens comprise co-administration of corticosteroids with a suspension of replication deficient recombinant adeno-associated virus (rAAV) comprising LDLR.

### 1. INTRODUCTION

The invention relates to a gene therapy for treating Familial Hypercholesterolemia (FH), and in particular, Homozygous FH (HoFH).

### 2. BACKGROUND OF THE INVENTION

Familial hypercholesterolemia (FH) is a life threatening disorder caused by mutations in genes that affect LDL receptor (LDLR) function (Goldstein et al. Familial hypercholesterolemia, in The Metabolic and Molecular Bases of Inherited Disease, C. R. Scriver, et al., Editors. 2001, McGraw-Hill Information Services Company: New York. p. 2863-2913 (2001)). It is estimated that >90% of patients with molecularly confirmed FH carry mutations in the gene encoding for the LDLR (LDLR, MIM 606945). The remainder of the patients carry mutations on three additional genes: APOB (MIM 107730) encoding apolipoprotein (apo) B, PCSK9 (MIM 607786) encoding proprotein convertase subtilisin/kexin type 9 (PCSK9), and LDLRAP1 (MIM 695747) encoding LDLR adapter protein 1. The latter is the only gene mutation that is associated with a recessive trait. Homozygosis is usually conferred by the presence of mutations in the 2 alleles of the same gene; however cases have been reported of patients with double heterozygosis (two heterozygous mutations, one each in two different genes). Based on prevalence rates of between 1 in 500 and 1 in 200 for heterozygous FH (Nordestgaard et al. Eur Heart J, 2013. 34(45): p. 3478-90a (2013), Sjouke et al. Eur Heart J, (2014)), it is estimated that between 7,000 and 43,000 people worldwide have homozygous FH (HoFH).

Characterization of mutant LDLR alleles has revealed a variety of mutations including deletions, insertions, missense mutations, and nonsense mutations (Goldstein et al. 2001). More than 1700 LDLR mutations have been reported. This genotypic heterogeneity leads to variable consequences in the biochemical function of the receptor which are classified in four general groups. Class 1 mutations are associated with no detectable protein and are often caused by gene deletions. Class 2 mutations lead to abnormalities in intracellular processing of the protein. Class 3 mutations specifically affect binding the ligand LDL, and Class 4 mutations encode receptor proteins that do not cluster in coated pits. Based on residual LDLR activity assessed using patients cultured fibroblasts, mutations are also classified as receptor negative (<2% residual activity of the LDLR) or receptor-defective (2-25% residual activity). Patients that are receptor-defective have, on average, lower LDL-C levels and a less malignant cardiovascular course.

As a consequence of impaired LDL receptor function, untreated total plasma cholesterol levels in patients with HoFH are typically greater than 500 mg/dl, resulting in premature and aggressive atherosclerosis often leading to cardiovascular disease (CVD) before age 20 and death before age 30 (Cuchel et al. Eur Heart J, 2014. 35(32): p. 2146-2157 (2014), Goldstein et al. 2001). Early initiation of aggressive treatment for these patients is therefore essential (Kolansky et al. 2008). The available options are limited. Statins are considered the first line for pharmacological treatment. Even at maximal doses, only a 10-25% reduction in LDL-C plasma levels is observed in most patients (Marais et al. Atherosclerosis, 2008. 197(1): p. 400-6 (2008); Raal et al. Atherosclerosis, 2000. 150(2): p. 421-8 (2000)). The addition of the cholesterol absorption inhibitor, ezetimibe, to statin therapy may result in a further 10-20% reduction in LDL-C levels (Gagne et al. Circulation, 2002. 105 (21): p. 2469-2475 (2002)). Use of other cholesterol lowering medications, including bile acid sequestrants, niacin, fibrates, and probucol have been used successfully in the pre-statin era and can be considered to achieve further LDL-C reduction in HoFH; however, their use is limited by tolerability and drug availability. This approach has been shown to reduce CVD and all-cause mortality (Raal et al. Circulation, 2011. 124(20): p. 2202-7). Despite the implementation of an aggressive multi-drug therapy approach, the LDL-C levels of HoFH patients remain elevated and their mean life expectancy remains approximately 32 years (Raal et al. 2011). Several non-pharmacological options have also been tested over the years. Surgical interventions, such as portacaval shunting (Bilheimer Arteriosclerosis, 1989. 9(1 Suppl): p. 1158-1163 (1989); Forman et al. Atherosclerosis, 1982. 41(2-3): p. 349-361 (1982)) and ileal bypass (Deckelbaum et al. N. Engl. J. Med. 1977; 296:465-470 1977. 296(9): p. 465-470 (1977)), have resulted only in partial and transient LDL-C lowering and are now considered nonviable approaches. Orthotopic liver transplantation has been demonstrated to substantially reduce LDL-C levels in HoFH patients (Ibrahim et al. J Cardiovasc Transl Res, 2012. 5(3): p. 351-8 (2012); Kucukkartallar et al. 2 Pediatr Transplant, 2011. 15(3): p. 281-4 (2011)), but disadvantages and risks limit the use of this approach, including the high risk of post-transplantation surgical complications and mortality, the scarcity of donors, and the need for life-long treatment with immunosuppressive therapy (Malatack Pediatr Transplant, 2011. 15(2): p. 123-5 (2011); Starzl et al. Lancet, 1984. 1(8391): p. 1382-1383 (1984)). The current standard of care in HoFH includes lipoprotein apheresis, a physical method of purging the plasma of LDL-C which can transiently reduce LDL-C by more than 50% (Thompson Atherosclerosis, 2003. 167(1): p. 1-13 (2003); Vella et al. Mayo Clin Proc, 2001. 76(10): p. 1039-46 (2001)). Rapid re-accumulation of LDL-C in plasma after treatment sessions (Eder and Rader Today's Therapeutic Trends, 1996. 14: p. 165-179 (1996)) necessitates weekly or biweekly apheresis. Although this procedure may delay the onset of atherosclerosis (Thompson et al. Lancet, 1995. 345: p. 811-816; Vella et al. Mayo Clin Proc, 2001. 76(10): p. 1039-46 (2001)), it is laborious, expensive, and not readily available. Furthermore, although it is a procedure that is generally well tolerated, the fact that it requires frequent repetition and intravenous access can be challenging for many HoFH patients.

Recently three new drugs have been approved by the FDA as add-on therapy specifically for HoFH. Two of them, lomitapide and mipomersen, inhibit the assembly and secretion of apoB-containing lipoproteins, although they do so via different molecular mechanisms (Cuchel et al. N Engl J Med, 2007. 356(2): p. 148-156 (2007); Raal et al. Lancet, 2010. 375(9719): p. 998-1006 (2010)). This approach results in a significant reduction of LDL-C that reaches an average of ˜50% with lomitapide (Cuchel et al. 2013) and ˜25% with mipomersen (Rall et al. 2010). However their use is associated with an array of adverse events that may affect tolerance and long term adherence and that include liver fat accumulation, the long term consequences of which have not yet been fully clarified.

The third is part of a novel class of lipid-lowering drugs, monoclonal antibodies against proprotein convertase subtilisin/kexin 9 (PCSK9) that have been shown to be effective in lowering LDL-C levels with an apparently favorable safety profile in patients with heterozygous FH (Raal et al. Circulation, 2012. 126(20): p. 2408-17 (2012), Raal et al. The Lancet, 2015. 385(9965): p. 341-350 (2015); Stein et al. Circulation, 2013. 128(19): p. 2113-20 (2012)). Treatment of HoFH with the PCSK9 inhibitor evolocumab 420 mg every 4 weeks for 12 weeks has been shown to provide about a 30% reduction in LDL-C as compared with placebo (Raal et al. 2015). Efficacy of PCSK9 inhibitors is, however, dependent on the residual LDLR activity, with no effect in patients with no residual LDLR activity (Raal et al. 2015, Stein et al. Circulation, 2013. 128(19): p. 2113-20 (2013)). Although the addition of PCSK9 inhibitors may become standard of care for FH and may provide an additional further reduction to lower hypercholesterolemia in a sub-set of HoFH patients, they will not dramatically impact the clinical management of this condition.

Therefore, there remains an unmet medical need for new medical therapies for HoFH.

### 3. SUMMARY OF THE INVENTION

A regimen comprising a replication deficient adeno-associated virus (AAV) to deliver a human Low Density Lipoprotein Receptor (hLDLR) gene to liver cells of patients (human subjects) diagnosed with HoFH is provided. The recombinant AAV vector (rAAV) used for delivering the LDLR gene (“rAAV.hLDLR”) should have a tropism for the liver (e.g., a rAAV bearing an AAV8 capsid), and the hLDLR transgene should be controlled by liver-specific expression control elements. Such rAAV.hLDLR vectors can be administered by intravenous (IV) infusion over a 20 to 30-minute period to achieve therapeutic levels of LDLR expression in the liver. In certain embodiments, the regimen comprises administering about 2.5×10¹³ genome copies (GC)/kg of the rAAV.hLDLR range. In certain embodiments, the regimen comprises co-administration of a tapering dose of steroid (e.g., equivalent to prednisone having an initial dose about 40 mg/day (or steroid equivalent). In certain embodiments, treating begins day −1 and continues to about week 8 post-dosing. In certain embodiments, the dose is tapered in a a 10 mg dose decrease/week for each of weeks 9 and 10, a 5 mg dose decrease/week for each of weeks 11, 12 and 13. In certain embodiments, the steroid regimen is also delivered when the patient receive does of about 2.5×10¹³ GC/kg to 7.5×10¹² genome copies, or other doses which are provided herein.

A regimen comprising a replication deficient adeno-associated virus (AAV) to deliver a human Low Density Lipoprotein Receptor (hLDLR) gene to liver cells of patients (human subjects) diagnosed with HoFH is provided. The recombinant AAV vector (rAAV) used for delivering the LDLR gene (“rAAV.hLDLR”) should have a tropism for the liver (e.g., a rAAV bearing an AAV8 capsid), and the hLDLR transgene should be controlled by liver-specific expression control elements. Such rAAV.hLDLR vectors can be administered by intravenous (IV) infusion over a 20 to 30-minute period to achieve therapeutic levels of LDLR expression in the liver. In certain embodiments, the regimen comprises administering about 2.5×10¹³ genome copies (GC)/kg of the rAAV.hLDLR range. In certain embodiments, the regimen comprises co-administration of a tapering dose of steroid (e.g., equivalent to prednisone having an initial dose about 40 mg/day (or steroid equivalent)). In certain embodiments, prophylactic co-treatment with steroid begins at least one day prior to gene therapy (day −1), or the day of gene therapy delivery (day 0), and continues to about week 8 post-dosing. In certain embodiments, prophylactic co-treatment begins at least one day prior or on the same day as gene therapy delivery and continues in a tapered dose to about week 13 post-dosing. Optionally, prophylactic steroid co-therapy may begin 2 or 3 days prior to vector dosing. In certain embodiments, the dose is tapered in a 10 mg dose decrease/week for each of weeks 9 and 10, a 5 mg dose decrease/week for each of weeks 11, 12 and 13. In certain embodiments, the prophylactic steroid regimen is also delivered when the patient receive lower doses (e.g., about 2.5×10¹² GC/kg to 7.5×10¹² GC/kg), or higher doses, such as provided herein.

The goal of the treatment is to functionally replace the patient's defective LDLR via rAAV-based liver-directed gene therapy as a viable approach to treat this disease and improve response to current lipid-lowering treatments. The invention is based, in part, on the development of therapeutic compositions and methods that allow for the safe delivery of efficacious doses; and improved manufacturing methods to meet the purification production requirement for efficacious dosing in human subjects.

Efficacy of the therapy may be assessed after treatment, e.g., post-dosing, using plasma LDL-C levels as a surrogate biomarker for human LDLR transgene expression in the patient. For example, a decrease in the patient's plasma LDL-C levels after the gene therapy treatment would indicate the successful transduction of functional LDLRs. Additionally, or alternatively, other parameters that can be monitored include, but are not limited to measuring changes in total cholesterol (TC), non-high density lipoprotein cholesterol (non-HDL-C), HDL-C, fasting triglycerides (TG), very low density lipoprotein cholesterol (VLDL-C), lipoprotein(a) (Lp(a)), apolipoprotein B (apoB), and apolipoprotein A-I (apoA-I) compared to baseline, as well as LDL kinetic studies (metabolic mechanism assessment) prior to vector and after vector administration, or combinations thereof.

In certain embodiments, efficacy of therapy may be measured by a reduction in the frequency of apheresis required by the patient. In certain embodiments, post-AAV8.hLDLR treatment, a patient may have his or her requirement for apheresis reduced by 25%, 50%, or more. For example, a patient receiving weekly apheresis prior to AAV8.hLDLR therapy may only require biweekly or monthly apheresis; in other embodiments, apheresis may be required even less frequently, or the need may be eliminated.

In certain embodiments, efficacy of therapy may be measured by a reduction in the dose of PCSK9 inhibitor required, or by an elimination of the need for such therapy in a patient post-AAV8.hLDLR treatment. In certain embodiments, efficacy of therapy is measured by a reduction in the dose of a statin or bile sequestrant required.

Patients who are candidates for treatment are preferably adults (male or female ≥18 years of age) diagnosed with HoFH carrying two mutations in the LDLR gene; i.e., patients that have molecularly defined LDLR mutations at both alleles in the setting of a clinical presentation consistent with HoFH, which can include untreated LDL-C levels, e.g., LDL-C levels >300 mg/dl, treated LDL-C levels, e.g., LDL-C levels <300 mg/dl and/or total plasma cholesterol levels greater than 500 mg/dl and premature and aggressive atherosclerosis. Candidates for treatment include HoFH patients that are undergoing treatment with lipid-lowering drugs, such as statins, ezetimibe, bile acid sequestrants, PCSK9 inhibitors, and LDL and/or plasma apheresis.

Prior to treatment, the HoFH patient should be assessed for neutralizing antibodies (NAb) to the AAV serotype used to deliver the hLDLR gene. Such NAbs can interfere with transduction efficiency and reduce therapeutic efficacy. HoFH patients that have a baseline serum NAb titer ≤1:10, are good candidates for treatment with the rAAV.hLDLR gene therapy protocol. However, patients with other baseline levels may be selected. Treatment of HoFH patients with titers of serum NAb >1:5 may require a combination therapy, such as transient co-treatment with an immunosuppressant before and/or during treatment with rAAV.hLDLR vector delivery. Additionally, or alternatively, patients are monitored for elevated liver enzymes, which may be treated with transient immunosuppressant therapy (e.g., if at least about 2× baseline levels of aspartate transaminase (AST) or alanine transaminase (ALT) are observed). Immunosuppressants for such co-therapy include, but are not limited to, steroids, antimetabolites, T-cell inhibitors, and alkylating agents.

The invention is illustrated by way of examples that describe a protocol for the AAV8.LDLR treatment of human subjects (Section 6, Example 1); pre-clinical animal data demonstrating efficacy of the treatment in animal models of disease (Section 7, Example 2); the manufacture and formulation of therapeutic AAV.hLDLR compositions (Sections 8.1 to 8.3, Example 3); and methods for characterization of the AAV vector (Section 8.4, Example 3).

### 3.1. Definitions

As used herein, “AAV8 capsid” refers to the AAV8 capsid having the encoded amino acid sequence of GenBank accession: YP_077180, which is incorporated by reference herein, and reproduced in SEQ ID NO: 5. Some variation from this encoded sequence is encompassed by the present invention, which may include sequences having about 99% identity to the referenced amino acid sequence in GenBank accession: YP_077180; U.S. Pat. Nos. 7,282,199, 7,790,449; 8,319,480; 8,962,330; 8,962,332, (i.e., less than about 1% variation from the referenced sequence). In another embodiment, the AAV8 capsid may have the VP1 sequence of the AAV8 variant described in WO2014/124282 or the dj sequence described in US 2013/0059732 A1 or U.S. Pat. No. 7,588,772 B2, which are incorporated by reference herein, which are incorporated by reference herein. Methods of generating the capsid, coding sequences therefore, and methods for production of rAAV viral vectors have been described. See, e.g., Gao, et al, Proc. Natl. Acad. Sci. U.S.A. 100 (10), 6081-6086 (2003), US 2013/0045186A1, and WO 2014/124282.

As used herein, the term “NAb titer” refers to a measurement of how much neutralizing antibody (e.g., anti-AAV NAb) is produced which neutralizes the physiologic effect of its targeted epitope (e.g., an AAV). Anti-AAV NAb titers may be measured as described in, e.g., Calcedo, R., et al., Worldwide Epidemiology of Neutralizing Antibodies to Adeno-Associated Viruses. Journal of Infectious Diseases, 2009. 199(3): p. 381-390, which is incorporated by reference herein.

The terms “percent (%) identity”, “sequence identity”, “percent sequence identity”, or “percent identical” in the context of amino acid sequences refers to the residues in the two sequences which are the same when aligned for correspondence. Percent identity may be readily determined for amino acid sequences over the full-length of a protein, polypeptide, about 32 amino acids, about 330 amino acids, or a peptide fragment thereof or the corresponding nucleic acid sequence coding sequencers. A suitable amino acid fragment may be at least about 8 amino acids in length, and may be up to about 700 amino acids. Generally, when referring to “identity”, “homology”, or “similarity” between two different sequences, “identity”, “homology” or “similarity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence. Alignments are performed using any of a variety of publicly or commercially available Multiple Sequence Alignment Programs. Sequence alignment programs are available for amino acid sequences, e.g., the “Clustal X”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27(13):2682-2690 (1999).

As used herein, the term “operably linked” refers to both expression control sequences that are contiguous with the gene of interest and expression control sequences that act in trans or at a distance to control the gene of interest.

A “replication-defective virus” or “viral vector” refers to a synthetic or artificial viral particle in which an expression cassette containing a gene of interest is packaged in a viral capsid or envelope, where any viral genomic sequences also packaged within the viral capsid or envelope are replication-deficient; i.e., they cannot generate progeny virions but retain the ability to infect target cells. In one embodiment, the genome of the viral vector does not include genes encoding the enzymes required to replicate (the genome can be engineered to be “gutless”—containing only the transgene of interest flanked by the signals required for amplification and packaging of the artificial genome), but these genes may be supplied during production. Therefore, it is deemed safe for use in gene therapy since replication and infection by progeny virions cannot occur except in the presence of the viral enzyme required for replication.

It is to be noted that the term “a” or “an” refers to one or more. As such, the terms “a” (or “an”), “one or more,” and “at least one” are used interchangeably herein.

The words “comprise”, “comprises”, and “comprising” are to be interpreted inclusively rather than exclusively. The words “consist”, “consisting”, and its variants, are to be interpreted exclusively, rather than inclusively. While various embodiments in the specification are presented using “comprising” language, under other circumstances, a related embodiment is also intended to be interpreted and described using “consisting of” or “consisting essentially of” language.

As used herein, the term “about” means a variability of 10% from the reference given, unless otherwise specified.

Unless defined otherwise in this specification, technical and scientific terms used herein have the same meaning as commonly understood by one of ordinary skill in the art and by reference to published texts, which provide one skilled in the art with a general guide to many of the terms used in the present application.

### 4. BRIEF DESCRIPTION OF THE DRAWINGS

FIGS. 1A-1H. Impact of pre-existing AAV8 NAb on EGFP expression levels in macaque livers. Macaques of different types and ages were injected via a peripheral vein with 3×10¹² GC/kg of AAV8.TBG.EGFP and were sacrificed 7 days later and analyzed for hepatocyte transduction in several ways. FIGS. 1A-1E are micrographs which show representative sections of liver from animals with various levels of pre-existing neutralizing antibodies to AAV8 (<1:5, 1:5, 1:10 and 1:20, respectively). FIG. 1F shows a quantitative morphometric analysis of the transduction efficiency based on percent transduction of hepatocytes. FIG. 1G shows quantitative morphometric analysis of the transduction efficiency based on relative EGFP intensity. FIG. 1H shows quantification of EGFP protein in liver lysate by ELISA. Adult cynomolgus macaques (n=8, closed circle), adult rhesus macaques (n=8, open triangle), juvenile rhesus macaques (n=5, open square).

FIG. 2. Long-term expression of mLDLR in DKO mice. DKO mice were dosed with 10¹¹ GC/mouse (5×10¹² GC/kg) of AAV8.TBG.mLDLR (n=10) or AAV8.TBG.nLacZ (n=10). Cholesterol levels in serum were monitored on a regular basis. Statistically significant differences between the two groups were realized as early as day 7 (p<0.001) and have remained throughout the duration of the experiment. Mice were sacrificed at day 180 after vector administration.

FIGS. 3A-3L. Regression of atherosclerosis in DKO mice following AAV8.TBG.mLDLR. FIG. 3A is a set of three panels with En face Sudan IV staining. Mouse aortas were pinned and stained with Sudan IV, which stains neutral lipids. Representative aortas from animals treated with 10¹¹ GC/mouse of AAV8.nLacZ (5×10¹² GC/kg) (middle), 10¹¹ GC/mouse of AAV8.TBG.mLDLR (5×10¹² GC/kg) (right) at day 60 after vector administration (day 120 on high fat diet), or at baseline (day 60 on high fat diet) (left) are shown. FIG. 3B is a bar chart showing the results of morphometric analyses quantified the percent of aorta stained with Oil Red O along the entire length of the aorta. FIGS. 3C-3K show the aortic roots from these mice were stained with Oil Red O. FIG. 3L is bar chart showing the percent Sudan IV staining of the total aortic surface in baseline (n=10), AAV.TBG.nLacZ (n=9), and AAV8.TBG.mLDLR (n=10) was determined. Quantification was conducted on Oil Red O lesions. Atherosclerotic lesion area data were subjected to a 1-way ANOVA. Experimental groups were compared with the baseline group by using the Dunnett test. Repeated-measures ANOVA was used to compare cholesterol levels among different groups of mice over time after gene transfer. Statistical significance for all comparisons was assigned at P, 0.05. Graphs represent mean SD values. *p<0.05, **p<0.01, ‡p<0.001.

FIG. 4. Cholesterol levels in test or control article injected DKO mice. DKO mice were injected IV with 7.5×10¹¹ GC/kg, 7.5×10¹² GC/kg or 6.0×10¹³ GC/kg of AAV8.TBG.mLDLR or 6.0×10¹³ GC/kg of AAV8.TBG.hLDLR or vehicle control (100 μl PBS). Cholesterol levels expressed as mean±SEM. Each group demonstrated a statistically significant reduction in serum cholesterol relative to PBS controls from the same necropsy time point.

FIGS. 5A-5B. Cholesterol levels in test article injected DKO mice. FIG. 5A shows cholesterol levels (mg/mL) in mice treated with varying doses of vector as measured on day 0, day 7 and day 30. Values expressed as mean±SEM. P<0.05.

FIGS. 6A-6C. Peripheral T cell responses in vector injected rhesus macaques. Data presented show the time course of T cell response and AST levels for macaques 19498 (FIG. 6A), 090-0287 (FIG. 6B), and 090-0263 (FIG. 6C). For each Study Day, T cell responses to no stimulation, AAV8 and hLDLR measured as spot-forming unit (SFU) per million PBMCs were plotted from left to right in each figure. Macaques 19498 and 090-0287 developed a positive peripheral T cell response to and/or the hLDLR transgene, whereas 090-0263 did not. * denotes positive capsid responses that were significantly above background.

FIG. 7. Schematic representation of AAV8.TBG.hLDLR vector.

FIGS. 8A-8B. AAV cis plasmid constructs. A) Linear representation of the paternal cis cloning plasmid, pENN.AAV.TBG.PI, containing the liver specific TBG promoter and chimeric intron flanked by AAV2 ITR elements. B) Linear representation of the human LDLR cis plasmid, pENN.AAV.TBG.PI.hLDLR.RBG.KanR, in which the human LDLR cDNA was cloned into pENN.AAV.TBG.PI between the intron and the poly A signal and the ampicillin resistance gene was replaced by the kanamycin resistance gene.

FIGS. 9A-9B. AAV trans plasmids. FIG. 9A is a Linear representation of the AAV8 trans packaging plasmid, p5E18-VD2/8, with the ampicillin resistance gene. FIG. 9B is a linear representation of the AAV8 trans packaging plasmid, pAAV2/8 with the kanamycin resistance gene.

FIGS. 10A-10B. Adenovirus helper plasmid. FIG. 10A illustrates derivation of the ad-helper plasmid, pAdΔF6, from the parental plasmid, pBHG10, through intermediates pAdΔF1 and pAdΔF5. FIG. 10B is a linear representation of the ampicillin resistance gene in pAdΔF6 was replaced by the kanamycin resistance gene to create pAdΔF6(Kan).

FIGS. 11A-11B. Flow Diagram showing AAV8.TBG.hLDLR vector manufacturing process.

### 5. DETAILED DESCRIPTION OF THE INVENTION

A replication deficient rAAV is used to deliver a hLDLR gene to liver cells of patients (human subjects) diagnosed with HoFH. The rAAV.hLDLR vector should have a tropism for the liver (e.g., an rAAV bearing an AAV8 capsid) and the hLDLR transgene should be controlled by liver-specific expression control elements.

Such rAAV.hLDLR vectors can be administered by intravenous (IV) infusion over about a 20 to about 30 minute period to achieve therapeutic levels of LDLR expression in the liver. In other embodiments, shorter (e.g., 10 to 20 minutes) or longer (e.g., over 30 minutes to 60 minutes, intervening times, e.g., about 45 minutes, or longer) may be selected. Therapeutically effective doses of the rAAV.hLDLR range from at least about 2.5×10¹² to 7.5×10¹² genome copies (GC)/kg body weight of the patient. In a preferred embodiment, the rAAV suspension has a potency such that a dose of 5×10¹¹ GC/kg administered to a double knockout LDLR−/−Apobec−/− mouse model of HoFH (DKO mouse) decreases baseline cholesterol levels in the DKO mouse by 25% to 75%. Efficacy of treatment can be assessed using Low density lipoprotein cholesterol (LDL-C) levels as a surrogate for transgene expression. Primary efficacy assessments include LDL-C levels at 1 to 3 months (e.g., week 12) post treatment, with persistence of effect followed thereafter for at least about 1 year (about 52 weeks). Long term safety and persistence of transgene expression may be measured post-treatment.

In certain embodiments, efficacy of therapy may be measured by a reduction in the frequency of apheresis required by the patient. In certain embodiments, post-AAV8.hLDLR treatment, a patient may have his or her requirement for apheresis reduced by 25%, 50%, or more. For example, a patient receiving weekly apheresis prior to AAV8.hLDLR therapy may only require biweekly or monthly apheresis; in other embodiments, apheresis may be required even less frequently or the need may be eliminated.

In certain embodiments, efficacy of therapy may be measured by a reduction in the dose of PCSK9 inhibitor required, or by an elimination of the need for such therapy in a patient post-AAV8.hLDLR treatment. In certain embodiments, efficacy of therapy is measured by a reduction in the dose of a statin or bile sequestrant required.

Patients who are candidates for treatment are preferably adults (male or female ≥18 years of age) diagnosed with HoFH carrying two mutations in the LDLR gene; i.e., patients that have molecularly defined LDLR mutations at both alleles in the setting of a clinical presentation consistent with HoFH, which can include untreated LDL-C levels, e.g., LDL-C levels >300 mg/dl, treated LDL-C levels, e.g., LDL-C levels <300 mg/dl and/or total plasma cholesterol levels greater than 500 mg/dl and premature and aggressive atherosclerosis. Candidates for treatment include HoFH patients that are undergoing treatment with lipid-lowering drugs, such as statins, ezetimibe, bile acid sequestrants, PCSK9 inhibitors, and LDL and/or plasma apheresis.

Prior to treatment, the HoFH patient should be assessed for neutralizing antibodies (NAb) to the AAV serotype used to deliver the hLDLR gene. Such NAbs can interfere with transduction efficiency and reduce therapeutic efficacy. HoFH patients that have a baseline serum NAb titer ≤1:10 are good candidates for treatment with the rAAV.hLDLR gene therapy protocol. Treatment of HoFH patients with titers of serum NAb >1:5 may require a combination therapy, such as transient co-treatment with an immunosuppressant before/during treatment with rAAV.hLDLR. Additionally, or alternatively, patients are monitored for elevated liver enzymes, which may be treated with transient immunosuppressant therapy (e.g., if at least about 2× baseline levels of aspartate transaminase (AST) or alanine transaminase (ALT) are observed).

In certain embodiments, such therapy may involve co-administration of two or more immunosuppressive drugs, the (e.g., prednelisone, micophenolate mofetil (MMF) and/or sirolimus (i.e., rapamycin)) on the same day. One or more of these drugs may be continued after gene therapy administration, at the same dose or an adjusted dose. Such therapy may be for about 1 week (7 days), about 60 days, or longer, as needed. In certain embodiments, a tacrolimus-free regimen is selected. additional immunosuppressant co-therapy is used. Immunosuppressants for such co-therapy include, but are not limited to, a glucocorticoid, steroids, antimetabolites, T-cell inhibitors, a macrolide (e.g., a rapamycin or rapalog), and cytostatic agents including an alkylating agent, an anti-metabolite, a cytotoxic antibiotic, an antibody, or an agent active on immunophilin. The immune suppressant may include a nitrogen mustard, nitrosourea, platinum compound, methotrexate, azathioprine, mercaptopurine, fluorouracil, dactinomycin, an anthracycline, mitomycin C, bleomycin, mithramycin, IL-2 receptor- (CD25-) or CD3-directed antibodies, anti-IL-2 antibodies, ciclosporin, tacrolimus, sirolimus, IFN-β, IFN-γ, an opioid, or TNF-α (tumor necrosis factor-alpha) binding agent. In certain embodiments, the immunosuppressive therapy may be started 0, 1, 2, 7, or more days prior to the gene therapy administration, or 0, 1, 2, 3, 7, or more days post-gene therapy administration.

Immunosuppressants for such co-therapy include, but are not limited to, a glucocorticoid, steroids, antimetabolites, T-cell inhibitors, a macrolide (e.g., a rapamycin or rapalog), and cytostatic agents including an alkylating agent, an anti-metabolite, a cytotoxic antibiotic, an antibody, or an agent active on immunophilin. The immune suppressant may include a nitrogen mustard, nitrosourea, platinum compound, methotrexate, azathioprine, mercaptopurine, fluorouracil, dactinomycin, an anthracycline, mitomycin C, bleomycin, mithramycin, IL-2 receptor- (CD25-) or CD3-directed antibodies, anti-IL-2 antibodies, ciclosporin, tacrolimus, sirolimus, IFN-β, IFN-γ, an opioid, or TNF-α (tumor necrosis factor-alpha) binding agent. In certain embodiments, the immunosuppressive therapy may be started 0, 1, 2, 7, or more days prior to the gene therapy administration, or 0, 1, 2, 3, 7, or more days post-gene therapy administration. Such therapy may involve co-administration of two or more drugs, the (e.g., prednelisone, micophenolate mofetil (MMF) and/or sirolimus (i.e., rapamycin)) on the same day. One or more of these drugs may be continued after gene therapy administration, at the same dose or an adjusted dose. Such therapy may be for about 1 week (7 days), about 60 days, or longer, as needed. In certain embodiments, a tacrolimus-free regimen is selected.

5.1 Gene Therapy Vectors

The rAAV.hLDLR vector should have a tropism for the liver (e.g., an rAAV bearing an AAV8 capsid) and the hLDLR transgene should be controlled by liver-specific expression control elements. The vector is formulated in a buffer/carrier suitable for infusion in human subjects. The buffer/carrier should include a component that prevents the rAAV, from sticking to the infusion tubing but does not interfere with the rAAV binding activity in vivo.

5.1.1. The rAAV.hLDLR Vector

Any of a number of rAAV vectors with liver tropism can be used. Examples of AAV which may be selected as sources for capsids of rAAV include, e.g., rh10, AAVrh64R1, AAVrh64R2, rh8 [See, e.g., US Published Patent Application No. 2007-0036760-A1; US Published Patent Application No. 2009-0197338-A1; EP 1310571]. See also, WO 2003/042397 (AAV7 and other simian AAV), U.S. Pat. Nos. 7,790,449 and 7,282,199 (AAV8), WO 2005/033321 and U.S. Pat. No. 7,906,111 (AAV9), WO 2006/110689 and WO 2003/042397 (rh10), AAV3B; US 2010/0047174 (AAV-DJ).

The hLDLR transgene can include, but is not limited to one or more of the sequences provided by SEQ ID NO:1, SEQ ID NO: 2, and/or SEQ ID NO: 4, which are provided in the attached Sequence Listing, which is incorporated by reference herein. With reference to SEQ ID NO:1, these sequences include a signal sequence located at about base pair 188 to about base pair 250 and the mature protein for variant 1 spans about base pair 251 to about base pair 2770. SEQ ID NO: 1 also identifies exons, at least one of which is absent in the known alternative splice variants of hLDLR. Additionally, or optionally, a sequence encoding one or more of the other hLDLR isoforms may be selected. See, e.g., isoforms 2, 3, 4, 5 and 6, the sequences of which are available, e.g., from uniprot.org/uniprot/P01130. For example, common variants lack exon 4 (bp (255) . . . (377) or exon 12 (bp (1546) . . . (1773)) of SEQ ID NO: 1). Optionally, the transgene may include the coding sequences for the mature protein with a heterologous signal sequence. SEQ ID NO: 2 provides the cDNA for human LDLR and the translated protein (SEQ ID NO: 3). SEQ ID NO: 4 provides an engineered cDNA for human LDLR. Alternatively or additionally, web-based or commercially available computer programs, as well as service based companies may be used to back translate the amino acids sequences to nucleic acid coding sequences, including both RNA and/or cDNA. See, e.g., backtranseq by EMBOSS, ebi.ac.uk/Tools/st/; Gene Infinity (geneinfinity.org/sms-/sms_backtranslation.html); ExPasy (expasy.org/tools/).

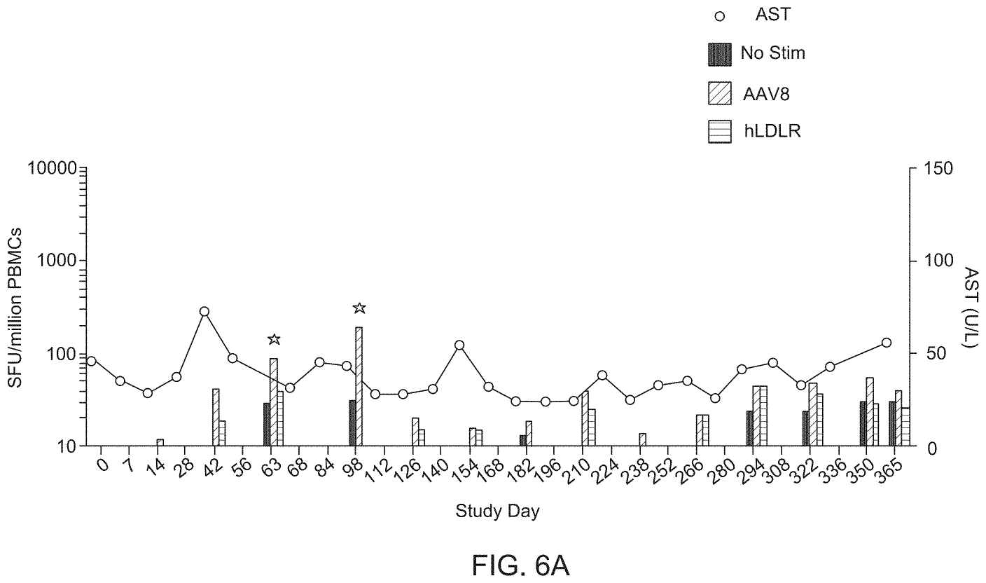

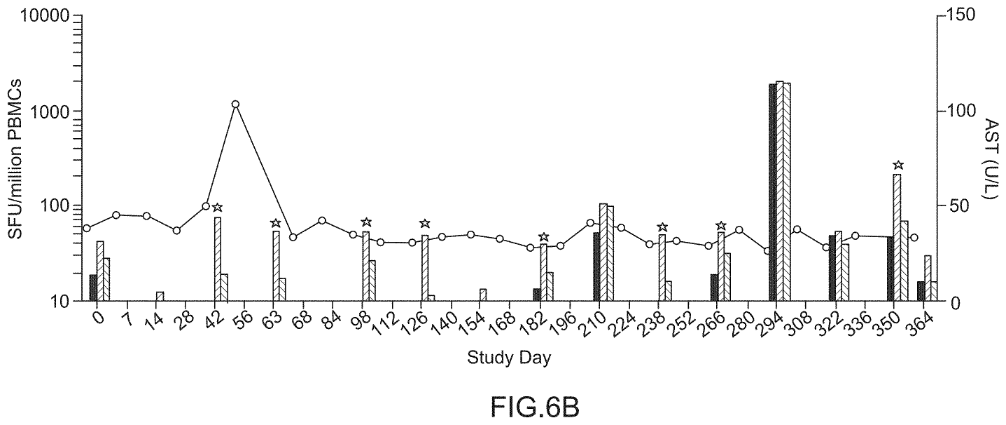

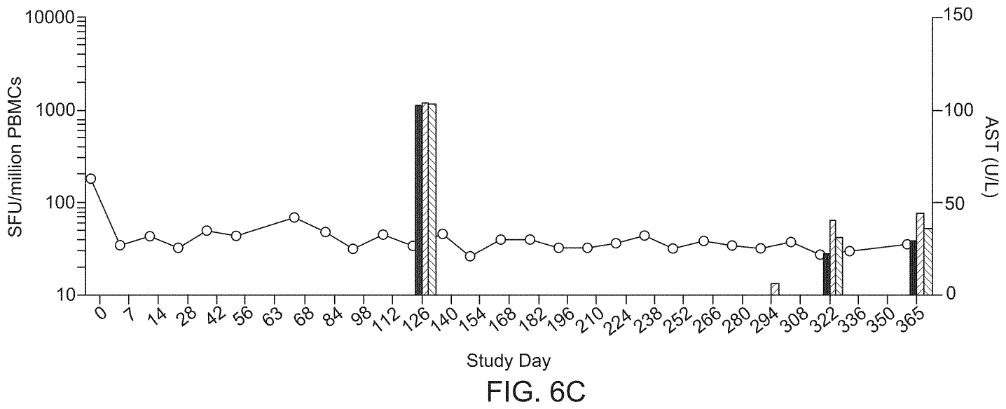

In a specific embodiment described in the Examples, infra, the gene therapy vector is an AAV8 vector expressing an hLDLR transgene under control of a liver-specific promoter (thyroxine-binding globulin, TBG) referred to as rAAV8.TBG.hLDLR (see FIG. 6). The external AAV vector component is a serotype 8, T=1 icosahedral capsid consisting of 60 copies of three AAV viral proteins, VP1, VP2, and VP3, at a ratio of 1:1:18. The capsid contains a single-stranded DNA rAAV vector genome.

The rAAV8.TBG.hLDLR genome contains an hLDLR transgene flanked by two AAV inverted terminal repeats (ITRs). The hLDLR transgene includes an enhancer, promoter, intron, an hLDLR coding sequence and polyadenylation (polyA) signal. The ITRs are the genetic elements responsible for the replication and packaging of the genome during vector production and are the only viral cis elements required to generate rAAV. Expression of the hLDLR coding sequence is driven from the hepatocyte-specific TBG promoter. Two copies of the alpha 1 microglobulin/bikunin enhancer element precede the TBG promoter to stimulate promoter activity. A chimeric intron is present to further enhance expression and a rabbit beta globin polyadenylation (polyA) signal is included to mediate termination of hLDLR mRNA transcripts.

An illustrative plasmid and vector described herein uses the liver-specific promoter thyroxin binding globulin (TBG). Alternatively, other liver-specific promoters may be used [see, e.g., The Liver Specific Gene Promoter Database, Cold Spring Harbor, http://rulai.schl.edu/LSPD, alpha 1 anti-trypsin (A1AT); human albumin Miyatake et al., J. Virol., 71:5124 32 (1997), humAlb; and hepatitis B virus core promoter, Sandig et al., Gene Ther., 3:1002 9 (1996)]. TTR minimal enhancer/promoter, alpha-antitrypsin promoter, LSP (845 nt)25(requires intron-less scAAV). Although less desired, other promoters, such as viral promoters, constitutive promoters, regulatable promoters [see, e.g., WO 2011/126808 and WO 2013/04943], or a promoter responsive to physiologic cues may be used may be utilized in the vectors described herein.

In addition to a promoter, an expression cassette and/or a vector may contain other appropriate transcription initiation, termination, enhancer sequences, efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. Examples of suitable polyA sequences include, e.g., SV40, bovine growth hormone (bGH), and TK polyA. Examples of suitable enhancers include, e.g., the alpha fetoprotein enhancer, the TTR minimal promoter/enhancer, LSP (TH-binding globulin promoter/alpha1-microglobulin/bikunin enhancer), amongst others.

These control sequences are “operably linked” to the hLDLR gene sequences.

The expression cassette may be engineered onto a plasmid which is used for production of a viral vector. The minimal sequences required to package the expression cassette into an AAV viral particle are the AAV 5′ and 3′ ITRs, which may be of the same AAV origin as the capsid, or which of a different AAV origin (to produce an AAV pseudotype). In one embodiment, the ITR sequences from AAV2, or the deleted version thereof (ΔITR), are used for convenience and to accelerate regulatory approval. However, ITRs from other AAV sources may be selected. Where the source of the ITRs is from AAV2 and the AAV capsid is from another AAV source, the resulting vector may be termed pseudotyped. Typically, an expression cassette for an AAV vector comprises an AAV 5′ ITR, the hLDLR coding sequences and any regulatory sequences, and an AAV 3′ ITR. However, other configurations of these elements may be suitable. A shortened version of the 5′ ITR, termed ΔITR, has been described in which the D-sequence and terminal resolution site (trs) are deleted. In other embodiments, the full-length AAV 5′ and 3′ ITRs are used.

The abbreviation “sc” refers to self-complementary. “Self-complementary AAV” refers a plasmid or vector having an expression cassette in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription. See, e.g., D M McCarty et al, “Self-complementary recombinant adeno-associated virus (scAAV) vectors promote efficient transduction independently of DNA synthesis”, Gene Therapy, (August 2001), Vol 8, Number 16, Pages 1248-1254. Self-complementary AAVs are described in, e.g., U.S. Pat. Nos. 6,596,535; 7,125,717 and 7,456,683, each of which is incorporated herein by reference in its entirety.

5.1.2. rAAV.hLDLR Formulation

The rAAV.hLDLR formulation is a suspension containing an effective amount of rAAV.hLDLR vector suspended in an aqueous solution containing buffering saline, a surfactant, and a physiologically compatible salt or mixture of salts adjusted to an ionic strength equivalent to about 100 mM sodium chloride (NaCl) to about 250 mM sodium chloride, or a physiologically compatible salt adjusted to an equivalent ionic concentration. In one embodiment, the formulation may contain, e.g., about 1.5×10¹¹ GC/kg to about 6×10¹³ GC/kg, or about 1×10¹² to about 1.25×10¹³ GC/kg, as measured by optimized qPCR (oqPCR) or digital droplet PCR (ddPCR) as described in, e.g., M. Lock et al, Hu Gene Therapy Methods, Hum Gene Ther Methods. 2014 April; 25(2):115-25. doi: 10.1089/hgtb.2013.131. Epub 2014 Feb. 14, which is incorporated herein by reference. For example, a suspension as provided herein may contain both NaCl and KCl. The pH may be in the range of 6.5 to 8, or 7 to 7.5. A suitable surfactant, or combination of surfactants, may be selected from among a Poloxamers, i.e., nonionic triblock copolymers composed of a central hydrophobic chain of polyoxypropylene (poly(propylene oxide)) flanked by two hydrophilic chains of polyoxyethylene (poly(ethylene oxide)), SOLUTOL HS 15 (Macrogol-15 Hydroxystearate), LABRASOL (Polyoxy capryllic glyceride), polyoxy 10 oleyl ether, TWEEN (polyoxyethylene sorbitan fatty acid esters), ethanol and polyethylene glycol. In one embodiment, the formulation contains a poloxamer. These copolymers are commonly named with the letter “P” (for poloxamer) followed by three digits: the first two digits×100 give the approximate molecular mass of the polyoxypropylene core, and the last digit×10 gives the percentage polyoxyethylene content. In one embodiment Poloxamer 188 is selected. The surfactant may be present in an amount up to about 0.0005% to about 0.001% of the suspension. In one embodiment, the rAAV.hLDLR formulation is a suspension containing at least 1×10¹³ genome copies (GC)/mL, or greater, as measured by oqPCR or digital droplet PCR (ddPCR) as described in, e.g., M. Lock et al, Hu Gene Therapy Methods, Hum Gene Ther Methods. 2014 April; 25(2):115-25. doi: 10.1089/hgtb.2013.131. Epub 2014 Feb. 14, which is incorporated herein by reference. In one embodiment, the vector is suspended in an aqueous solution containing 180 mM sodium chloride, 10 mM sodium phosphate, 0.001% Poloxamer 188, pH 7.3. The formulation is suitable for use in human subjects and is administered intravenously. In one embodiment, the formulation is delivered via a peripheral vein by infusion over 20 minutes (±5 minutes). However, this time may be adjusted as needed or desired.

In order to ensure that empty capsids are removed from the dose of AAV. hLDLR that is administered to patients, empty capsids are separated from vector particles during the vector purification process, e.g., using cesium chloride gradient ultracentrifugation as discussed in detail herein at Section 8.3.2.5. In one embodiment, the vector particles containing packaged genomes are purified from empty capsids using the process described in International Patent Application No. PCT/US16/65976, filed Dec. 9, 2016, U.S. Patent Appln No. 62/322,093, filed Apr. 13, 2016 and U.S. Patent Appln No. 62/266,341, filed on Dec. 11, 2015, and entitled “Scalable Purification Method for AAV8”, which is incorporated by reference herein. Briefly, a two-step purification scheme is described which selectively captures and isolates the genome-containing rAAV vector particles from the clarified, concentrated supernatant of a rAAV production cell culture. The process utilizes an affinity capture method performed at a high salt concentration followed by an anion exchange resin method performed at high pH to provide rAAV vector particles which are substantially free of rAAV intermediates. In certain embodiments, the method separates recombinant AAV8 viral particles containing DNA comprising pharmacologically active genomic sequences from genome-deficient(empty) AAV8 capsid intermediates. The method involves (a) forming a loading suspension comprising: recombinant AAV8 viral particles and empty AAV8 capsid intermediates which have been purified to remove non-AAV materials from an AAV producer cell culture in which the particles and intermediates were generated; and a Buffer A comprising 20 mM Bis-Tris propane (BTP) and a pH of about 10.2; (b) loading the suspension of (a) onto a strong anion exchange resin, said resin being in a vessel having an inlet for flow of a suspension and/or solution and an outlet permitting flow of eluate from the vessel; (c) washing the loaded anion exchange resin with Buffer 1% B which comprises 10 mM NaCl and 20 mM BTP with a pH of about 10.2; (d) applying an increasing salt concentration gradient to the loaded and washed anion exchange resin, wherein the salt gradient ranges from 10 mM to about 190 mM NaCl, inclusive of the endpoints, or an equivalent; and (e) collecting the rAAV particles from eluate, said rAAV particles being purified away from intermediates.

In one embodiment, the pH used is from 10 to 10.4 (about 10.2) and the rAAV particles are at least about 50% to about 90% purified from AAV8 intermediates, or a pH of 10.2 and about 90% to about 99% purified from AAV8 intermediates. In one embodiment, this is determined by genome copies. A stock or preparation of rAAV8 particles (packaged genomes) is “substantially free” of AAV empty capsids (and other intermediates) when the rAAV8 particles in the stock are at least about 75% to about 100%, at least about 80%, at least about 85%, at least about 90%, at least about 95%, or at least 99% of the rAAV8 in the stock and “empty capsids” are less than about 1%, less than about 5%, less than about 10%, less than about 15% of the rAAV8 in the stock or preparation.

In one embodiment, the formulation is characterized by an rAAV stock having a ratio of “empty” to “full” of 1 or less, preferably less than 0.75, more preferably, 0.5, preferably less than 0.3.

In a further embodiment, the average yield of rAAV particles is at least about 70%. This may be calculated by determining titer (genome copies) in the mixture loaded onto the column and the amount presence in the final elutions. Further, these may be determined based on q-PCR analysis and/or SDS-PAGE techniques such as those described herein or those which have been described in the art.

For example, to calculate empty and full particle content, VP3 band volumes for a selected sample (e.g., an iodixanol gradient-purified preparation where # of GC=# of particles) are plotted against GC particles loaded. The resulting linear equation (y=mx+c) is used to calculate the number of particles in the band volumes of the test article peaks. The number of particles (pt) per 20 μL loaded is then multiplied by 50 to give particles (pt)/mL. Pt/mL divided by GC/mL gives the ratio of particles to genome copies (pt/GC). Pt/mL-GC/mL gives empty pt/mL. Empty pt/mL divided by pt/mL and ×100 gives the percentage of empty particles.

Generally, methods for assaying for empty capsids and AAV vector particles with packaged genomes have been known in the art. See, e.g., Grimm et al., Gene Therapy (1999) 6:1322-1330: Sommer et al., Molec. Ther. (2003) 7:122-128. To test for denatured capsid, the methods include subjecting the treated AAV stock to SDS-polyacrylamide gel electrophoresis, consisting of any gel capable of separating the three capsid proteins, for example, a gradient gel containing 3-8% Tris-acetate in the buffer, then running the gel until sample material is separated, and blotting the gel onto nylon or nitrocellulose membranes, preferably nylon. Anti-AAV capsid antibodies are then used as the primary antibodies that bind to denatured capsid proteins, preferably an anti-AAV capsid monoclonal antibody. most preferably the B1 anti-AAV-2 monoclonal antibody (Wobus et al., J Virol. (2000) 74:9281-9293). A secondary antibody is then used, one that binds to the primary antibody and contains a means for detecting binding with the primary antibody, more preferably an anti-IgG antibody containing a detection molecule covalently bound to it, most preferably a sheep anti-mouse IgG antibody covalently linked to horseradish peroxidase. A method for detecting binding is used to semi-quantitatively determine binding between the primary and secondary antibodies, preferably a detection method capable of detecting radioactive isotope emissions, electromagnetic radiation, or colorimetric changes, most preferably a chemiluminescence detection kit. For example, for SDS-PAGE, samples from column fractions can be taken and heated in SDS-PAGE loading buffer containing reducing agent (e.g., DTT), and capsid proteins were resolved on pre-cast gradient polyacrylamide gels (e.g., Novex). Silver staining may be performed using SilverXpress (Invitrogen, CA) according to the manufacturer's instructions. In one embodiment, the concentration of AAV vector genomes (vg) in column fractions can be measured by quantitative real time PCR (Q-PCR). Samples are diluted and digested with DNase I (or another suitable nuclease) to remove exogenous DNA. After inactivation of the nuclease, the samples are further diluted and amplified using primers and a TaqMan™ fluorogenic probe specific for the DNA sequence between the primers. The number of cycles required to reach a defined level of fluorescence (threshold cycle, Ct) is measured for each sample on an Applied Biosystems Prism 7700 Sequence Detection System. Plasmid DNA containing identical sequences to that contained in the AAV vector is employed to generate a standard curve in the Q-PCR reaction. The cycle threshold (Ct) values obtained from the samples are used to determine vector genome titer by normalizing it to the Ct value of the plasmid standard curve. End-point assays based on the digital PCR can also be used.

In one aspect, an optimized q-PCR method is provided herein which utilizes a broad spectrum serine protease, e.g., proteinase K (such as is commercially available from Qiagen). More particularly, the optimized qPCR genome titer assay is similar to a standard assay, except that after the DNase I digestion, samples are diluted with proteinase K buffer and treated with proteinase K followed by heat inactivation. Suitably samples are diluted with proteinase K buffer in an amount equal to the sample size. The proteinase K buffer may be concentrated to 2-fold or higher. Typically, proteinase K treatment is about 0.2 mg/mL, but may be varied from 0.1 mg/mL to about 1 mg/mL. The treatment step is generally conducted at about 55° C. for about 15 minutes, but may be performed at a lower temperature (e.g., about 37° C. to about 50° C.) over a longer time period (e.g., about 20 minutes to about 30 minutes), or a higher temperature (e.g., up to about 60° C.) for a shorter time period (e.g., about 5 to 10 minutes). Similarly, heat inactivation is generally at about 95° C. for about 15 minutes, but the temperature may be lowered (e.g., about 70 to about 90° C.) and the time extended (e.g., about 20 minutes to about 30 minutes). Samples are then diluted (e.g., 1000 fold) and subjected to TaqMan analysis as described in the standard assay.

Additionally, or alternatively, droplet digital PCR (ddPCR) may be used. For example, methods for determining single-stranded and self-complementary AAV vector genome titers by ddPCR have been described. See, e.g., M. Lock et al, Hu Gene Therapy Methods, Hum Gene Ther Methods. 2014 April; 25(2):115-25. doi: 10.1089/hgtb.2013.131. Epub 2014 Feb. 14.

5.1.3 Manufacturing

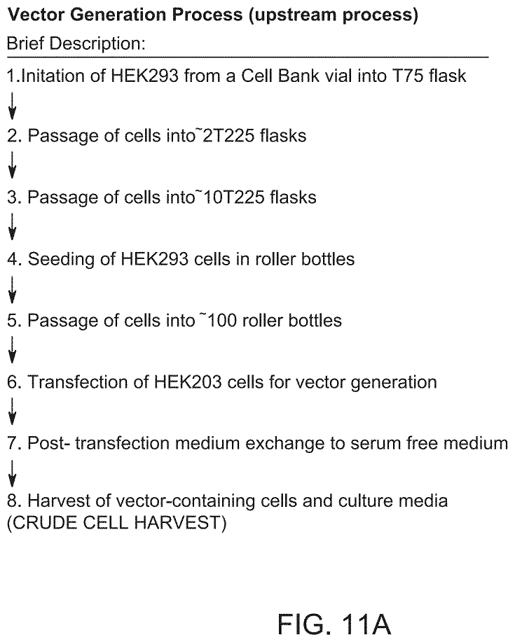

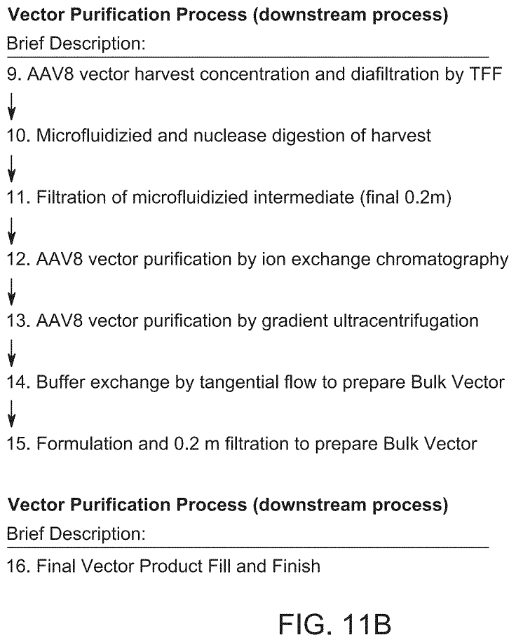

The rAAV.hLDLR vector can be manufactured as shown in the flow diagram shown in FIG. 11. Briefly, cells (e.g. HEK 293 cells) are propagated in a suitable cell culture system and transfected for vector generation. The rAAV.hLDLR vector can then be harvested, concentrated and purified to prepare bulk vector which is then filled and finished in a downstream process.

Methods for manufacturing the gene therapy vectors described herein include methods well known in the art such as generation of plasmid DNA used for production of the gene therapy vectors, generation of the vectors, and purification of the vectors. In some embodiments, the gene therapy vector is an AAV vector and the plasmids generated are an AAV cis-plasmid encoding the AAV genome and the gene of interest, an AAV trans-plasmid containing AAV rep and cap genes, and an adenovirus helper plasmid. The vector generation process can include method steps such as initiation of cell culture, passage of cells, seeding of cells, transfection of cells with the plasmid DNA, post-transfection medium exchange to serum free medium, and the harvest of vector-containing cells and culture media. The harvested vector-containing cells and culture media are referred to herein as crude cell harvest.

The crude cell harvest may thereafter be subject method steps such as concentration of the vector harvest, diafiltration of the vector harvest, microfluidization of the vector harvest, nuclease digestion of the vector harvest, filtration of microfluidized intermediate, purification by chromatography, purification by ultracentrifugation, buffer exchange by tangential flow filtration, and formulation and filtration to prepare bulk vector.

In certain embodiments, methods similar to those of FIG. 11 may be used in conjunction with other AAV producer cells. Numerous methods are known in the art for production of rAAV vectors, including transfection, stable cell line production, and infectious hybrid virus production systems which include Adenovirus-AAV hybrids, herpesvirus-AAV hybrids and baculovirus-AAV hybrids. See, e.g., G Ye, et al, Hu Gene Ther Clin Dev, 25: 212-217 (December 2014); R M Kotin, Hu Mol Genet, 2011, Vol. 20, Rev Issue 1, R2-R6; M. Mietzsch, et al, Hum Gene Therapy, 25: 212-222 (March 2014); T Virag et al, Hu Gene Therapy, 20: 807-817 (August 2009); N. Clement et al, Hum Gene Therapy, 20: 796-806 (August 2009); DL Thomas et al, Hum Gene Ther, 20: 861-870 (August 2009). rAAV production cultures for the production of rAAV virus particles all require; 1) suitable host cells, including, for example, human-derived cell lines such as HeLa, A549, or 293 cells, or insect-derived cell lines such as SF-9, in the case of baculovirus production systems; 2) suitable helper virus function, provided by wild type or mutant adenovirus (such as temperature sensitive adenovirus), herpes virus, baculovirus, or a nucleic acid construct providing helper functions in trans or in cis; 3) functional AAV rep genes, functional cap genes and gene products; 4) a transgene (such as a therapeutic transgene) flanked by AAV ITR sequences; and 5) suitable media and media components to support rAAV production.

A variety of suitable cells and cell lines have been described for use in production of AAV. The cell itself may be selected from any biological organism, including prokaryotic (e.g., bacterial) cells, and eukaryotic cells, including, insect cells, yeast cells and mammalian cells. Particularly desirable host cells are selected from among any mammalian species, including, without limitation, cells such as A549, WEHI, 3T3, 10T1/2, BHK, MDCK, COS 1, COS 7, BSC 1, BSC 40, BMT 10, VERO, WI38, HeLa, a HEK 293 cell (which express functional adenoviral E1), Saos, C2C12, L cells, HT1080, HepG2 and primary fibroblast, hepatocyte and myoblast cells derived from mammals including human, monkey, mouse, rat, rabbit, and hamster. In certain embodiments, the cells are suspension-adapted cells. The selection of the mammalian species providing the cells is not a limitation of this invention; nor is the type of mammalian cell, i.e., fibroblast, hepatocyte, tumor cell, etc.

In a specific embodiment, the methods used for manufacturing the gene therapy vectors are described in Example 3 at Section 8, infra.

5.2 Patient Population

Patients who are candidates for treatment are preferably adults (male or female ≥18 years of age) diagnosed with HoFH carrying two mutations in the LDLR gene; i.e., patients that have molecularly defined LDLR mutations at both alleles in the setting of a clinical presentation consistent with HoFH, which can include untreated LDL-C levels, e.g., LDL-C levels >300 mg/dl, treated LDL-C levels, e.g., LDL-C levels <300 mg/dl and/or total plasma cholesterol levels greater than 500 mg/dl and premature and aggressive atherosclerosis. In some embodiments, a patient <18 years of age can be treated. In some embodiments, the patient that is treated is a male ≥18 years of age. In some embodiments, the patient that is treated is a female ≥18 years of age. Candidates for treatment include HoFH patients that are undergoing treatment with lipid-lowering drugs, such as statins, ezetimibe, bile acid sequestrants, PCSK9 inhibitors, and LDL and/or plasma apheresis.

Prior to treatment, the HoFH patient should be assessed for NAb to the AAV serotype used to deliver the hLDLR gene. Such NAbs can interfere with transduction efficiency and reduce therapeutic efficacy. HoFH patients that have a baseline serum NAb titer ≤1:10 are good candidates for treatment with the rAAV.hLDLR gene therapy protocol. However, patients with higher ratios may be selected under certain circumstances. Treatment of HoFH patients with titers of serum NAb >1:5 may require a combination therapy, such as transient co-treatment with an immunosuppressant, although such therapy may be selected for patients with lower ratios. Immunosuppressants for such co-therapy include, but are not limited to, steroids, antimetabolites, T-cell inhibitors, and alkylating agents. For example, such transient treatment may include a steroid (e.g., prednisole) dosed once daily for 7 days at a decreasing dose, in an amount starting at about 60 mg, and decreasing by 10 mg/day (day 7 no dose). Other doses and medications may be selected.

Subjects may be permitted to continue their standard of care treatment(s) (e.g., LDL apheresis and/or plasma exchange, and other lipid lowering treatments) prior to and concurrently with the gene therapy treatment at the discretion of their caring physician. In the alternative, the physician may prefer to stop standard of care therapies prior to administering the gene therapy treatment and, optionally, resume standard of care treatments as a co-therapy after administration of the gene therapy. Desirable endpoints of the gene therapy regimen are low density lipoprotein cholesterol (LDL-C) reduction and change in fractional catabolic rate (FCR) of LDL apolipoprotein B (apoB) from baseline up to 12 weeks after administration of the gene therapy treatment. Other desired endpoints include, e.g., reduction in one or more of: total cholesterol (TC), non-high density lipoprotein cholesterol (non-HDL-C), decrease in fasting triglycerides (TG), and changes in HDL-C (e.g., increased levels are desirable), very low density lipoprotein cholesterol (VLDL-C), lipoprotein(a) (Lp(a)), apolipoprotein B (apoB), and/or apolipoprotein A-I (apoA-I).

In one embodiment, patients achieve desired LDL-C thresholds (e.g., LDL-C<200, <130, or <100, mg/dl) after treatment with AAV8.hLDLR, alone and/or combined with the use of adjunctive treatments over the duration of the study.

In certain embodiments, patients will have a reduced need for lipid lowering therapy, including frequency of LDL and/or plasma apheresis.

In still other embodiments, there will be a reduction in number, size or extent of assessable xanthomas compared to baseline.

Nevertheless, patients having one or more of the following characteristics may be excluded from treatment at the discretion of their caring physician:
Heart failure defined by the NYHA classification as functional Class III with history of hospitalization(s) within 12 weeks of the baseline visit or functional Class IV.
History within 12 weeks of the baseline visit of a myocardial infarction (MI), unstable angina leading to hospitalization, coronary artery bypass graft surgery (CABG), percutaneous coronary intervention (PCI), uncontrolled cardiac arrhythmia, carotid surgery or stenting, stroke, transient ischemic attack, carotid revascularization, endovascular procedure or surgical intervention.
Uncontrolled hypertension defined as: systolic blood pressure >180 mmHg, diastolic blood pressure >95 mmHg.
History of cirrhosis or chronic liver disease based on documented histological evaluation or non-invasive imaging or testing.
Documented diagnosis of any of the following liver diseases: Nonalcoholic steatohepatitis (biopsy-proven); Alcoholic liver disease; Autoimmune hepatitis; Liver cancer; Primary biliary cirrhosis; Primary sclerosing cholangitis; Wilson's disease; Hemochromatosis; α₁ anti-trypsin deficiency.
Abnormal LFTs at screening (AST or ALT >2× upper limit of normal (ULN) and/or Total Bilirubin of >1.5×ULN unless patient has unconjugated hyperbilirubinemia due to Gilbert's syndrome).
Hepatitis B as defined by positive for HepB SAg, or Hep B Core Ab, and/or viral DNA, or Chronic active Hepatitis C as defined by positive for HCV Ab and viral RNA.
History of alcohol abuse within 52 weeks.
Certain prohibited medications known to be potentially hepatotoxic, especially those that can induce microvesicular or macrovesicular steatosis. These include but are not limited to: acutane, amiodarone, HAART medications, heavy acetaminophen use (2 g/day >3×q week), isoniazid, methotrexate, tetracyclines, tamoxifen, valproate.
Active tuberculosis, systemic fungal disease, or other chronic infection.
History of immunodeficiency diseases, including a positive HIV test result.
Chronic renal insufficiency defined as estimated GRF <30 mL/min.
History of cancer within the past 5 years, except for adequately treated basal cell skin cancer, squamous cell skin cancer, or in situ cervical cancer.
Previous organ transplantation.
Any major surgical procedure occurring less than 3 months prior to determination of baselines and/or treatment.

A baseline serum AAV8 NAb titer >1:5, >1:10. In other embodiments, a caring physician may determine that the presence of one or more of these physical characteristics (medical history) should not preclude treatment as provided herein.

5.3. Dosing & Route of Administration

Patients receive a single dose of rAAV.hLDLR administered, e.g., via a peripheral vein by infusion; e.g., over about 20 to about 30 minutes. The dose of rAAV.hLDLR administered to a patient is 2.5×10¹³ GC/kg (as measured by oqPCR or ddPCR).

In certain embodiments, prophylactic immunomodulatory co-treatment with steroid begins at least one day prior to gene therapy (day −1), or the day of gene therapy delivery (day 0), and continues to about week 8 post-dosing. In certain embodiments, prophylactic co-treatment begins at least one day prior or on the same day as gene therapy delivery and continues in a tapered dose to about week 13 post-dosing. Optionally, prophylactic steroid co-therapy may begin 2 or 3 days prior to vector dosing. In certain embodiments, the dose is tapered in a 10 mg dose decrease/week for each of weeks 9 and 10, a 5 mg dose decrease/week for each of weeks 11, 12 and 13. In certain embodiments, the prophylactic steroid regimen is also delivered when the patient receive lower doses (e.g., about 2.5×1012 GC/kg to 7.5×1012 GC/kg), or higher doses, such as provided herein. In certain embodiments, another corticosteroid may be substituted for prednisone. In such an instance, a corticosteroid dose equivalent is provided. For example, suitable alternatives to 40 mg prednisone may include, e.g., betamethasone (about 6 mg), cortisone (about 200 mg), dexamethasone (about 6 mg), hydrocortisone (160 mg), methylprednisolone (about 32 mg), prednisolone (about 40 mg), or triamcinolone (about 32 mg). Other immunomodulators and dose equivalents may be determined.

In certain embodiments, the dose beginning on the day prior to dosing (Day −1). The starting dose is prednisone 40 mg once daily with a taper beginning at Week 9 and continuing through the end of Week 13. The first dose should be given on Day −1 at least 8 hours before scheduled dosing with study vector.

Prednisone Dose by Study Week

| | Week(s) | | | | | | |
|---|---|---|---|---|---|---|---|
| | Day −1 to | Week | Week | Week | Week | Week | Week |
| | Week 8 | 9 | 10 | 11 | 12 | 13 | 14 |
| Daily | 40 | 30 | 20 | 15 | 10 | 5 | 0 |
| Prednisone | | | | | | | |
| dose | | | | | | | |
| (mg/day)* | | | | | | | |

In certain embodiments, patients receive a co-therapy comprising at least 2.5×10¹² GC/kg or 7.5×10¹² GC/kg, or at least 5×10¹¹ GC/kg to about 7.5×10¹² GC/kg (as measured by oqPCR or ddPCR) in co-therapy with prednisone or a dose equivalent corticosteroid. However, other doses may be selected.

Optionally, additional immunomodulators may be utilized in this regimen. In certain embodiments, such additional immunomodulators are introduced post-dosing.

In a preferred embodiment, the rAAV suspension used has a potency such that a dose of 5×10¹¹ GC/kg administered to a double knockout LDLR−/−Apobec−/− mouse model of HoFH (DKO mouse) decreases baseline cholesterol levels in the DKO mouse by 25% to 75%.

In some embodiments, the dose of rAAV.hLDLR administered to a patient is in the range of 2.5×10¹² GC/kg to 7.5×10¹² GC/kg. Preferably, the rAAV suspension used has a potency such that a dose of 5×10¹¹ GC/kg administered to a double knockout LDLR−/−Apobec−/− mouse model of HoFH (DKO mouse) decreases baseline cholesterol levels in the DKO mouse by 25% to 75%. In specific embodiments, the dose of rAAV.hLDLR administered to a patient is at least 5×10¹¹ GC/kg 2.5×10¹² GC/kg, 3.0×10¹² GC/kg, 3.5×10¹² GC/kg, 4.0×10¹² GC/kg, 4.5×10¹² GC/kg, 5.0×10¹² GC/kg, 5.5×10¹² GC/kg, 6.0×10¹² GC/kg, 6.5×10¹² GC/kg, 7.0×10¹² GC/kg, or 7.5×10¹² GC/kg.

In some embodiments, rAAV.hLDLR is administered in combination with one or more therapies for the treatment of HoFH. In some embodiments, rAAV.hLDLR is administered in combination with standard lipid-lowering therapy that is used to treat HoFH, including but not limited to statin, ezetimibe, ezedia, bile acid sequestrants, LDL apheresis, plasma apheresis, plasma exchange, lomitapide, mipomersen, and/or PCSK9 inhibitors. In some embodiments, rAAV.hLDLR is administered in combination with niacin. In some embodiments, rAAV.hLDLR is administered in combination with fibrates.

5.4. Measuring Clinical Objectives

Safety of the gene therapy vector after administration can be assessed by the number of adverse events, changes noted on physical examination, and/or clinical laboratory parameters assessed at multiple time points up to about 52 weeks post vector administration. Although physiological effect may be observed earlier, e.g., in about 1 day to one week, in one embodiment, steady state levels expression levels are reached by about 12 weeks.

LDL-C reduction achieved with rAAV.hLDLR administration can be assessed as a defined percent change in LDL-C at about 12 weeks, or at other desired time points, compared to baseline.

Other lipid parameters can also be assessed at about 12 weeks, or at other desired time points, compared to baseline values, specifically percent change in total cholesterol (TC), non-high density lipoprotein cholesterol (non-HDL-C), HDL-C, fasting triglycerides (TG), very low density lipoprotein cholesterol (VLDL-C), lipoprotein(a) (Lp(a)), apolipoprotein B (apoB), and apolipoprotein A-I (apoA-I). The metabolic mechanism by which LDL-C is reduced can be assessed by performing LDL kinetic studies prior to rAAV.hLDLR administration and again 12 weeks after administration. The primary parameter to be evaluated is the fractional catabolic rate (FCR) of LDL apoB.

As used herein, the rAAV.hLDLR vector herein “functionally replaces” or “functionally supplements” the patients defective LDLR with active LDLR when the patient expresses a sufficient level of LDLR to achieve at least one of these clinical endpoints. Expression levels of hLDLR which achieve as low as about 10% to less than 100% of normal wild-type clinical endpoint levels in a non-FH patient may provide functional replacement.

In one embodiment, expression may be observed as early as about 8 hours to about 24 hours post-dosing. One or more of the desired clinical effects described above may be observed within several days to several weeks post-dosing.

Long term (up to 260 weeks) safety and efficacy can be assessed after rAAV.hLDLR administration.

Standard clinical laboratory assessments and other clinical assays described in Sections 6.4.1 through 6.7 infra, can be used to monitor adverse events, efficacy endpoints that assess percent change in lipid parameters, pharmacodynamic assessments, lipoprotein kinetics, ApoB-100 concentrations, as well as immune responses to the rAAV.hLDLR vector.

The following examples are illustrative only and are not intended to limit the present invention.

### EXAMPLES

### 6. Example 1: Protocol for Treating Human Subjects

This Example relates to a gene therapy treatment for patients with genetically confirmed homozygous familial hypercholesterolemia (HoFH) due to mutations in the low density lipoprotein receptor (LDLR) gene. In this example, the gene therapy vector, AAV8.TBG.hLDLR, a replication deficient adeno-associated viral vector 8 (AAV8) expressing hLDLR is administered to patients with HoFH. Efficacy of treatment can be assessed using Low density lipoprotein cholesterol (LDL-C) levels as a surrogate for transgene expression. Primary efficacy assessments include LDL-C levels at about 12 weeks post treatment, with persistence of effect followed thereafter for at least 52 weeks. Long term safety and persistence of transgene expression may be measured post-treatment in liver biopsy samples.

6.1. Gene Therapy Vector

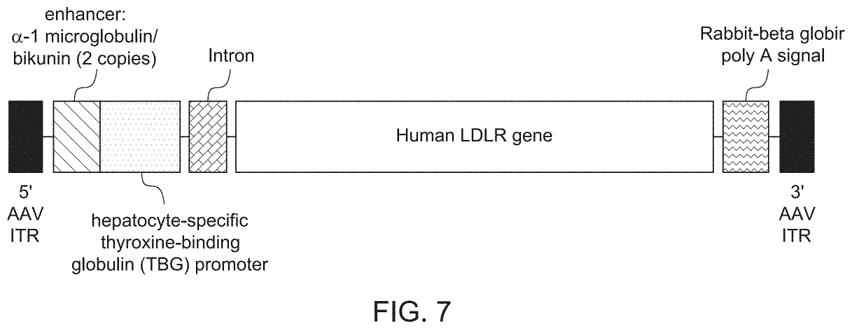

The gene therapy vector is an AAV8 vector expressing the transgene human low density lipoprotein receptor (hLDLR) under control of a liver-specific promoter (thyroxine-binding globulin, TBG) and is referred to in this Example as AAV8.TBG.hLDLR (see FIG. 7). The AAV8.TBG.hLDLR vector consists of the AAV vector active ingredient and a formulation buffer. The external AAV vector component is a serotype 8, T=1 icosahedral capsid consisting of 60 copies of three AAV viral proteins, VP1, VP2, and VP3, at a ratio of 1:1:18. The capsid contains a single-stranded DNA recombinant AAV (rAAV) vector genome. The genome contains an hLDLR transgene flanked by two AAV inverted terminal repeats (ITRs). An enhancer, promoter, intron, hLDLR coding sequence and polyadenylation (polyA) signal comprise the hLDLR transgene. The ITRs are the genetic elements responsible for the replication and packaging of the genome during vector production and are the only viral cis elements required to generate rAAV. Expression of the hLDLR coding sequence is driven from the hepatocyte-specific TBG promoter. Two copies of the alpha 1 microglobulin/bikunin enhancer element precede the TBG promoter to stimulate promoter activity. A chimeric intron is present to further enhance expression and a rabbit beta globin polyadenylation (polyA) signal is included to mediate termination of hLDLR mRNA transcripts. The sequence of pAAV.TBG.PI.hLDLRco.RGB which was used to produce this vector is provided in SEQ ID NO: 6.

The formulation of the investigational agent is at least 1×10¹³ genome copies (GC)/mL in aqueous solution containing 180 mM sodium chloride, 10 mM sodium phosphate, 0.001% Poloxamer 188, pH 7.3 and is administered via a peripheral vein by infusion over 20 minutes (±5 minutes).

6.2. Patient Population

Patients treated are adults with homozygous familial hypercholesterolemia (HoFH) carrying two mutations in the LDLR gene. The patients can be males or females that are 18 years old or older. The patients have molecularly defined LDLR mutations at both alleles in the setting of a clinical presentation consistent with HoFH, which can include untreated LDL-C levels, e.g., LDL-C levels >300 mg/dl, treated LDL-C levels, e.g., LDL-C levels <300 mg/dl and/or total plasma cholesterol levels greater than 500 mg/dl and premature and aggressive atherosclerosis. The treated patients can be concurrently undergoing treatment with lipid-lowering drugs, such as statins, ezetimibe, bile acid sequestrants, PCSK9 inhibitors, and LDL apheresis and/or plasma apheresis.

Patients that are treated can have a baseline serum AAV8 neutralizing antibody (NAb) titer ≤1:10. If a patient does not have a baseline serum AAV8 neutralizing antibody (NAb) titer ≤1:10, the patient can be transiently co-treated with an immunosuppressant during the transduction period. In certain embodiments, a patient with an AAV8 neutralizing antibody titer may be higher (e.g., ≤1:5 to ≤1:15, or ≤1:20) or lower (e.g., ≤1:2 to ≤1:5). Immunosuppressants for co-therapy include, but are not limited to, steroids, antimetabolites, T-cell inhibitors, and alkylating agents.

Subjects may be permitted to continue their standard of care treatment(s) (e.g., LDL apheresis and/or plasma exchange, and other lipid lowering treatments) prior to and concurrently with the gene therapy treatment at the discretion of their caring physician. In the alternative, the physician may prefer to stop standard of care therapies prior to administering the gene therapy treatment and, optionally, resume standard of care treatments as a co-therapy after administration of the gene therapy. Desirable endpoints of the gene therapy regimen are low density lipoprotein cholesterol (LDL-C) reduction and change in fractional catabolic rate (FCR) of LDL apolipoprotein B (apoB) from baseline up to about 12 weeks after administration of the gene therapy treatment.

In still other embodiments, desirable endpoints include reduction in the need for LDL apheresis and/or plasma apheresis is a desirable endpoint. The term “LDL apheresis” is used to refer to low-density lipoprotein (LDL) apheresis which is a process in which LDL is eliminated from the bloodstream using a process similar to dialysis. LDL apheresis is a procedure that removes LDL cholesterol from the blood of patients. During the LDL-apheresis procedure, the blood cells are separated from the plasma. Specialized filters are used to remove the LDL cholesterol from the plasma, and the filtered blood is returned to the patient. A single LDL apheresis treatment can remove 60-70% of harmful LDL cholesterol from the blood. There are currently two machines that are approved in the U.S by the Food and Drug Administration. The Liposorber uses a filter covered with dextran, which attaches to the LDL and removes it from the circulation. The other machine is called HELP and uses heparin to remove the LDL. Neither of these machines causes significant changes in the amount of HDL (good) cholesterol. These are currently approved for patients with LDL cholesterol of 2000 ng/mdl or higher with a history of coronary artery disease and patients with LDL cholesterol levels of 300 mg/dl or higher without coronary artery disease. See, e.g., American Society for Apheresis, www.apheresis.com, and http://c_ymcdn.com/sites/www.apheresis.org/resource/resmgr/-fact_sheets_file/ldl_apberesis.pdf. See, also, World Apheresis Association [http://worldaphersis.org/] and The National Lipid Association (USA) [https://www.lipid.org/]. In certain embodiments, plasma apheresis (plasmapheresis) which is unselective for LDL may have been used prior to gene therapy treatment and the need for such treatment may be reduced as described herein for LDL apheresis. As used herein, “reduction” in apheresis refers to a decrease in the number of times a month and/or a year which a patient is required to undergo apheresis. Such a reduction may be 10%, 25%, 50%, 75%, or 100% (e.g., eliminating the need) less apheresis treatments post-therapy as compared to the level of apheresis used prior to the rAAV8-hLDLR therapy. For example, a selected patient who had been undergoing apheresis weekly pre-treatment with rAAV8.hLDLR may only require apheresis every two weeks, monthly, or less frequently post-treatment. In another example, a selected patient who had been undergoing apheresis twice a month pre-treatment with rAAV8.hLDLR may only require apheresis every monthly, bi-monthly, quarterly or less frequently post-treatment. Still other

In certain embodiments, a desirable endpoint includes reduction in the dose of a PCSK9 inhibitor used to treat the patient is a desirable endpoint. As used herein, “reduction” in apheresis refers to a decrease in the number of times a month and/or a year which a patient is required to undergo apheresis. Such a reduction may be 10%, 25%, 50%, 75%, or 100% (e.g., eliminating the need) less PCSK9 inhibitor required post-therapy as compared to the level of PCSK9 inhibitor used prior to the rAAV8-hLDLR therapy. For example, treating a HoFH patient on a PCSK9 inhibitor pre-rAAV8.hLDLR therapy (e.g., receiving 300 mg-500 mg dose) once a month by infusion, may result in the ability to reduce treatment with the PCSK9 inhibitor to a treatment level consistent with a HeFH patient. This may result in the patient being able to receive less intrusive therapy (e.g., eliminating the need for infusion of high doses). For example, rather than a monthly infusion of 420 mg/by infusion, the patient may be electable for administration of a lower dose with a syringe or autoinjector (e.g., 100-140 ng/mL) once a month or every two weeks (HeFH dose), or less frequently.

6.3. Dosing & Route of Administration

Patients receive a single dose of AAV8.TBG.hLDLR administered via a peripheral vein by infusion. The dose of AAV8. TBG.hLDLR administered to a patient is about 2.5×10¹² GC/kg or 7.5×10² GC/kg. In order to ensure that empty capsids are removed from the dose of AAV8.TBG.hLDLR that is administered to patients, empty capsids are separated from vector particles by cesium chloride gradient ultracentrifugation or by ion exchange chromatography during the vector purification process, as discussed in Section 8.3.2.5.

6.4. Measuring Clinical Objectives
LDL-C reduction achieved with AAV8.TBG.hLDLR administration can be assessed as a defined percent change in LDL-C at about 12 weeks compared to baseline.
Other lipid parameters can be assessed at about 12 weeks compared to baseline values, specifically percent change in total cholesterol (TC), non-high density lipoprotein cholesterol (non-HDL-C), HDL-C, fasting triglycerides (TG), very low density lipoprotein cholesterol (VLDL-C), lipoprotein(a) (Lp(a)), apolipoprotein B (apoB), and apolipoprotein A-I (apoA-I).
The metabolic mechanism by which LDL-C is reduced can be assessed by performing LDL kinetic studies prior to vector administration and again at about 12 weeks after administration. The primary parameter to be evaluated is the fractional catabolic rate (FCR) of LDL apoB.
Long term (up to 52 weeks or up to 260 weeks) safety and efficacy can be assessed after AAV8.TBG.hLDLR administration

6.4.1. Standard Clinical Laboratory Assessments that can be Performed:

The following clinical profiles can be tested before and after treatment:
Biochemical Profile: sodium, potassium, chloride, carbon dioxide, glucose, blood urea nitrogen, lactate dehydrogenase (LDH) creatinine, creatinine phosphokinase, calcium, total protein, albumin, aspartate aminotransferase (AST), alanine aminotransferase (ALT), alkaline phosphatase, total bilirubin, GGT.
CBC: white blood cell (WBC) count, hemoglobin, hematocrit, platelet count, red cell distribution width, mean corpuscular volume, mean corpuscular hemoglobin, and mean corpuscular hemoglobin concentration.
Coagulation: PT, INR, PTT (at screening and baseline, and as needed.
Urinalysis: urinary color, turbidity, pH, glucose, bilirubin, ketones, blood, protein, WBC's.

6.4.2. Adverse Events of Interest

The following clinical assays can be used to monitor toxicity:
Liver injury
   CTCAE v4.0 grade 3 or higher lab result for bilirubin or liver enzymes (AST, ALT, AlkPhos).
   Bilirubin and AlkPhos CTCAE v4.0 grade 2 (bilirubin >1.5×ULN; AlkPhos >2.5×ULN).
Hepatotoxicity (i.e., meet criteria for “Hy's law”)
   ≥3×ULN (Upper limit of normal) for AST or ALT, and
   >2×ULN serum total bilirubin without elevated alkaline phosphatase, and
   No other reason can be found to explain the increased transaminase levels combined with increased total bilirubin.
      Additionally, ALT or AST elevations that may trigger corticosteroid therapy for presumed T-cell mediated immune transaminitis (>2× baseline AND 1×ULN) will be flagged and reported.

6.5. Efficacy Endpoints

Assessment of the percent change in lipid parameters at about 12 weeks following administration of AAV8.TBG.hLDLR can be assessed and compared to baseline. This includes:
Percent changes in LDL-C directly measured (primary efficacy endpoint).
Percent changes in Total Cholesterol, VLDL-C, HDL-C, calculated non-HDL-cholesterol, Changes in triglycerides, apoA-I, apoB, and Lp(a).

Baseline LDL-C value can be calculated as the average of LDL-C levels obtained under fasting condition in 2 separate occasions before administration of AAV8.TBG.hLDLR to control for laboratory and biological variability and ensure a reliable efficacy assessment.

6.5.1. Pharmacodynamic/Efficacy Assessments

The following efficacy laboratory tests can be evaluated under fasting conditions:
LDL-C directly measured
Lipid panel: total cholesterol, LDL-C, non-HDL-C, HDL-C, TG, Lp(a)
Apolipoproteins: apoB and apoA-I.

Additionally, optional LDL apoB kinetics may be determined prior to and 12 weeks after treatment. Lipid lowering efficacy may be assessed as percent changes from baseline at about 12, 24 and 52 weeks post vector administration. Baseline LDL-C values are calculated by averaging the LDL-C levels obtained under fasting condition in 2 separate occasions before administration. The percent change from baseline in LDL-C at 12 weeks post vector administration is the primary measure of gene transfer efficacy.
Change in LDL-apoB fractional catabolic rate from baseline to 12 weeks after vector administration. Additional apoB kinetic parameters will be also considered.
Absolute LDL-C levels at 12 weeks, 24 weeks, 52 weeks and annually up to 260 weeks following administration of AAV8.hLDLR.
Percent change in LDL-C and other lipid parameters from baseline at 24 weeks, 52 weeks and annually up to 260 weeks following administration of AAV8. hLDLR
The percentage of subjects who achieve absolute LDL-C levels <200 mg/dl at 12 weeks, 24 weeks, 52 weeks following administration of AAV8.hLDLR.
The number of subjects at 12 weeks, 24 weeks, 36 weeks, 52 weeks who did not resume previously taken or did not initiate any new lipid lowering treatment, following administration of AAV8.hLDLR.
For those subjects who received lipid apheresis prior to screening, the number of subjects who experienced a change in frequency of apheresis treatments any time during the study.
For those subjects who received a PCSK9 inhibitor, the LDL-C achieved following administration of AAV8. hLDLR compared with the LDL-C achieved while on the PCSK9 inhibitor prior to administration of AAV8.hLDLR.
For subjects with easy to describe xanthomas at baseline, the number who have documented improvement in number, size or extent of clinical presentation at 12 weeks and 52 weeks following administration of AAV8.hLDLR.

6.6. Lipoprotein Kinetics

Lipoprotein kinetic studies may be performed prior to vector administration and again 12 weeks after to assess the metabolic mechanism by which LDL-C is reduced. The primary parameter to be evaluated is the fractional catabolic rate (FCR) of LDL-apoB. Endogenous labeling of apoB is achieved by intravenous infusion of deuterated leucine, followed by blood sampling over a 48 hour period.

6.6.1. ApoB-100 Isolation

VLDL, IDL and LDL are isolated by sequential ultracentrifugation of timed samples drawn after the D3-leucine infusion. Apo B-100 is isolated from these lipoproteins by preparative sodium dodecyl sulfate-polyacrylamide gel electrophoresis (SDS PAGE) using a Tris-glycine buffer system. ApoB concentrations within individual apoB species are determined by enzyme-linked immunosorbent assay (ELISA). The total apoB concentration is determined using an automated immunoturbidimetric assay.

6.6.2. Isotopic Enrichment Determinations

ApoB-100 bands are excised from polyacrylamide gels. Excised bands are hydrolyzed in 12N HCl at 100° C. for 24 hours. Amino acids are converted to the N-isobutyl ester and N-heptafluorobutyramide derivatives before analysis using a gas chromatograph/mass spectrometer. Isotope enrichment (percentage) is calculated from the observed ion current ratios. Data in this format are analogous to specific radioactivity in radiotracer experiments. It is assumed that each subject remains in steady state with respect to apoB-100 metabolism during this procedure.

6.7. Pharmacokinetics and Immune Response to AAV8 Assessments

The following tests can be used to evaluate pharmacokinetics, pre-immunity to the AAV vector and immune response to the AAV vector:
Immune response monitoring: AAV8 NAb titer; T-cell responses to AAV8 vector; T-cell responses to hLDLR.
Vector concentration: AAV8 concentrations in plasma, measured as vector genomes by PCR.
Human Leukocyte Antigen Typing (HLA type): HLA type is assessed in deoxyribonucleic acid (DNA) from peripheral blood mononuclear cells (PBMCs) by high resolution evaluation of HLA-A, HLA-B, HLA-C for Class I and HLA DRB1/DRB345, DQB1 and DPB1 for Class II. This information allows for correlation of the potential T cell immune response to AAV8 capsid or to LDLR transgene with a specific HLA allele, helping to explain individual variability in the intensity and timing of T cell responses.

6.8 Xanthoma Assessment

Physical exams include identification, examination and description of any xanthomas. Documentation of xanthoma location and type is determined, i.e., cutaneous, palpebral (eye), tuberous, and/or tendinous. Where possible, metric rulers or calipers are used to document size of xanthomas (largest and smallest extents) during physical exam. If possible, digital photographs of xanthomas that are most extensive and readily identifiable are made with placement of a tape ruler (metric with millimeters) next to the lesion.

### 7. Example 2: Pre-Clinical Data

Nonclinical studies were undertaken to study the effects of AAV8.TBG.hLDLR on animal models for HoHF and pre-existing humoral immunity. Multiple single dose pharmacology studies were conducted in small and large animal models measuring decreases in cholesterol. Additionally, regression in atherosclerosis was measured in the Double Knock-Out LDLR−/−Apobec1−/− mouse model (DKO), which is deficient in both LDLR and Apobec1, develops severe hypercholesterolemia due to elevations in apoB-100-containing LDL even on a chow diet, and develops extensive atherosclerosis. These data were used to determine a minimally effective dose and to adequately justify dose selection for human studies. To further characterize the appropriate dose for human studies and identify potential safety signals, toxicology studies were conducted in non-human primates (NHPs) and a mouse model of HoFH.

7.1 Pre-Existing Humoral Immunity: Effect on AAV-Mediated Gene Transfer to Liver

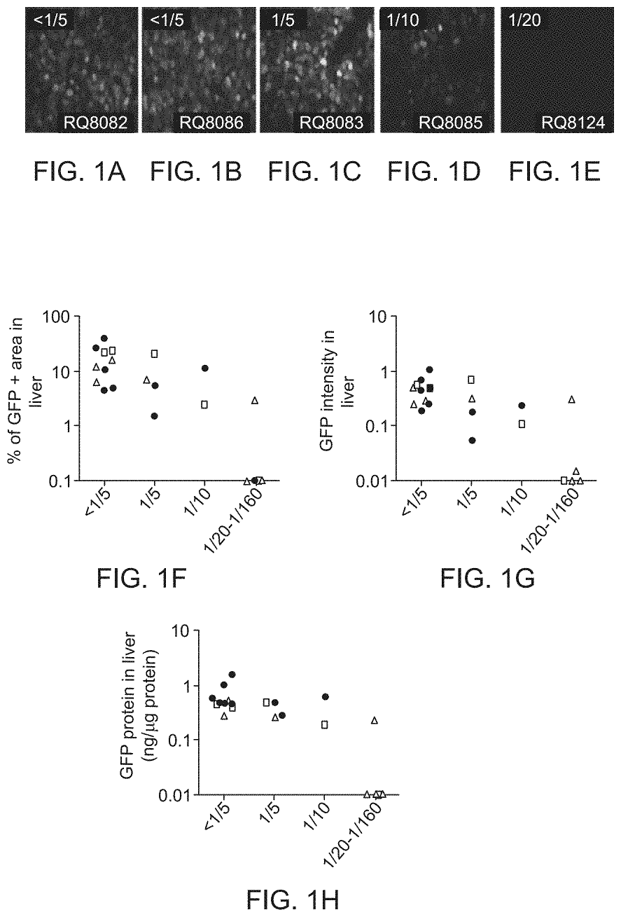

The goal of this study was to evaluate the impact of pre-existing humoral immunity to AAV on liver directed gene transfer using AAV8 encapsidated vectors in rhesus and cynomolgus macaques. Twenty-one rhesus and cynomolgus macaques were selected from a larger population of animals who were pre-screened for levels of pre-existing immunity against AAV8. Animals represented a wide age distribution and all were male. These studies focused on animals with low to undetectable levels of neutralizing antibodies (NAbs) while including a more limited number with AAV8 NAb titers up to 1:160. Animals were infused with 3×10² GC/kg of AAV8 vector expressing enhanced green fluorescent protein (EGFP) from the liver-specific tyroxine binding globulin (TBG) promoter via a peripheral vein infusion. Animals were necropsied 7 days later and tissues were evaluated for EGFP expression and liver targeting of AAV8 vector genomes (FIG. 1). Pre-existing NAbs to AAV8 in NHP sera were assessed using an in vitro transduction inhibition assay, as well as in the context of passive transfer experiments, in which sera from NHP was infused into mice prior to and at the time of vector administration to evaluate the impact of pre-existing AAV8 NAbs on liver directed gene transfer in vivo (Wang et al., 2010 Molecular Therapy 18(1): 126-134).

Animals with undetectable to low levels of pre-existing NAbs to AAV8 displayed high level transduction in liver, as evidenced by EGFP detection by fluorescent microscopy (FIG. 1) and ELISA, as well as vector DNA quantification in the liver. The most useful measure of transduction in terms of efficacy in HoFH is percent of hepatocytes transduced, which in the absence of pre-existing NAb was 17% (range of 4.4% to 40%). This is very close to the efficiency observed in mice at the same dose of vector. T threshold titer of pre-existing NAbs significantly impacting transduction of liver cells was ≤1:5 (i.e., titers of 1:10 or greater substantially reduced transduction). Antibody-mediated inhibition of liver transduction correlated directly with diminished AAV genomes in liver. Human sera were screened for evidence of pre-existing NAb to AAV8 and results suggest that about 15% of adults have NAbs to AAV8 that are in excess of ≤1:5. Also, it was shown that higher levels of NAb are associated with a change in the biodistribution of the vector, such that NAb decreases liver gene transfer while increasing deposition of the vector genome into the spleen, without increasing spleen transduction.

7.2 Effect of AAV8.TBG.mLDLR on Serum Cholesterol in a Mouse Model of HoFH

DKO mice (6 to 12 week old males) were injected IV with AAV8.TBG.mLDLR and followed for metabolic correction and reversal of pre-existing atherosclerosis lesions. Animals were also evaluated for gross clinical toxicity and abnormalities in serum transaminases. The mouse version of LDLR was utilized for vector administration into the DKO mouse.

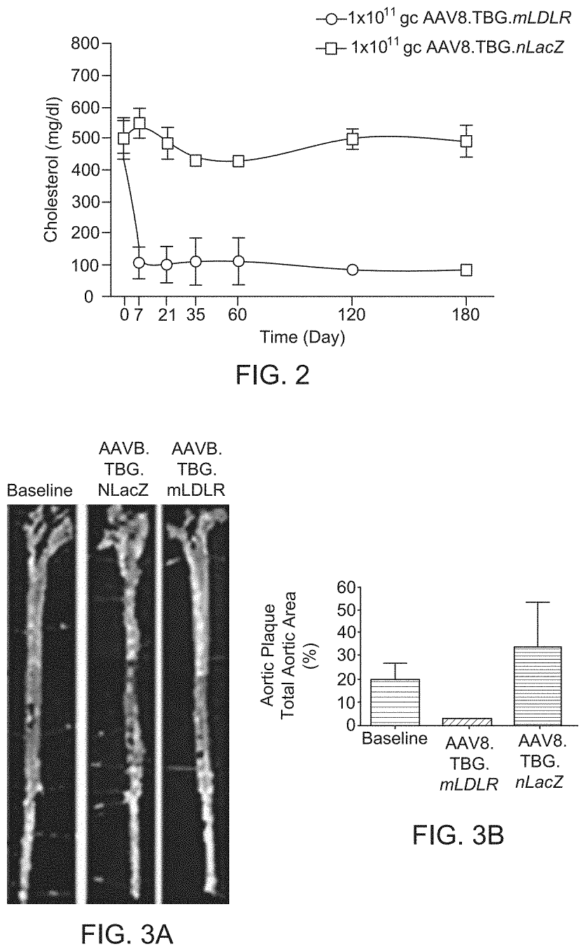

Mice that received 10¹¹ GC/mouse (5×10¹² GC/kg) showed a near complete normalization of hypercholesterolemia that was stable for 180 days (FIG. 2). No elevation in ALT levels or abnormal liver biochemistry were observed for up to 6 months post-vector injection at the highest doses in rodents (Kassim et al., 2010, PLoS One 5(10): e13424).

7.3 Effect of AAV8.TBG.mLDLR on Atherosclerotic Lesions in a Mouse Model of HoFH on a High-Fat Diet

Given that AAV8-mediated delivery of LDLR induced significant lowering of total cholesterol, AAV8-mediated expression of mLDLR was examined in a proof-of-concept study to determine whether it had an effect on atherosclerotic lesions (Kassim et al., 2010, PLoS One 5(10): e13424). Three groups of male DKO mice were fed a high-fat diet to hasten the progression of atherosclerosis. After two months, one group of mice received a single IV injection of 5×10¹² GC/kg of control AAV8.TBG.nLacZ vector, one group received a single IV injection of 5×10¹² GC/kg of AAV8.TBG.mLDLR vector, while a third non-intervention group were necropsied for atherosclerosis lesion quantification. The mice which received vectors were maintained on the high-fat diet for an additional 60 days at which time they were necropsied.

Animals that received the AAV8.TBG.mLDLR vector realized a rapid drop in total cholesterol from 1555±343 mg/dl at baseline to 266±78 mg/dl at day 7 and to 67±13 mg/dl by day 60 after treatment. By contrast, the plasma cholesterol levels of AAV8.TBG.nLacZ treated mice remained virtually unchanged from 1566±276 mg/dl at baseline to 1527±67 mg/dl when measured 60 days after vector. All animals developed slight increases in serum transaminases following the two months on the high-fat diet, which remained elevated following treatment with the AAV8.TBG.nLacZ vector but diminished three-fold to normal levels after treatment with the AAV8. TBG.mLDLR vector.

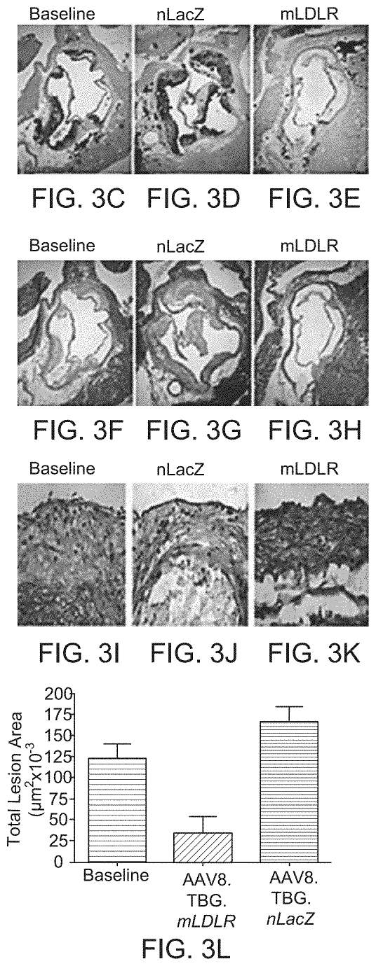

Evolution of pre-existing atherosclerotic lesions was assessed by two independent methods. In the first method the aortas were opened from the arch to the iliac bifurcation and stained with Oil Red O (FIG. 3A); morphometric analyses quantified the percent of aorta stained with Oil Red O along the entire length of the aorta (FIG. 3B). Oil Red O is a lysochrome (fat-soluble dye) diazo dye used for staining of neutral triglycerides and lipids on frozen sections. Staining of the aorta with this dye allows for the visualization of lipid laden plaques. As seen in FIG. 3, two months of high fat diet resulted in extensive atherosclerosis covering 20% of the aorta reflecting the baseline disease at the time of vector; this increased to 33% over an additional two month period following treatment with the AAV8.TBG.nLacZ vector, representing a 65% further progression in atherosclerosis. In contrast, treatment with the AAV8.TBG.mLDLR vector led to a regression of atherosclerosis by 87% over two months, from 20% of the aorta covered by atherosclerosis at baseline to only 2.6% of the aorta covered by atherosclerosis 60 days after vector administration.

In the second method, total lesion area was quantified in the aortic root (FIG. 3C-F). This analysis revealed the same overall trends, with AAV8.TBG.nLacZ injected mice showing a 44% progression over 2 months compared to baseline mice, while AAV8.TBG.mLDLR injected mice demonstrating a 64% regression in lesion compared with baseline mice. In summary, expression of LDLR via injection of AAV8.TBG.mLDLR induced marked reduction in cholesterol and substantial regression of atherosclerosis over two months as assessed by two independent methods of quantification at two different sites within the aorta.

7.4 Assessment of Minimal Effective Dose in a Mouse Model of HoFH

Extensive studies of the correlations between phenotypes and genotype in HoFH populations have demonstrated that differences in LDL and total cholesterol of only 25-30% translate to substantial differences in clinical outcome (Bertolini et al. 2013, Atherosclerosis 227(2): 342-348; Kolansky et al. 2008, Am J Cardiol 102(11): 1438-1443; Moorjani et al. 1993, The Lancet 341(8856): 1303-1306). Furthermore, lipid-lowering treatment associated with LDL-C reduction lower than 30%, translates to delayed cardiovascular events and prolonged survival in patients with HoFH (Raal et al. 2011, Circulation 124(20): 2202-2207). Recently, the FDA approved the drug mipomersen for the treatment of HoFH in which the primary endpoint was a reduction of LDL-C of 20 to 25% from baseline (Raal et al. 2010, Lancet 375(9719): 998-1006).

Against this background, the minimal effective dose (MED) in the gene therapy mouse studies discussed below was defined as the lowest dose of vector that lead to a statistically significant and stable reduction of total cholesterol in the serum that is at least 30% lower than baseline. The MED has been evaluated in a number of different studies and a brief description of each experiment is provided below.

7.4.1. POC Dose-Ranging Study of AAV8.TBG.mLDLR in DKO Mice

A proof-of-concept dose-ranging study of AAV8.TBG.mLDLR and AAV8.TBG.hLDLR in DKO mice was conducted to identify suitable doses for further study. In these studies, DKO male mice were injected IV with different doses of AAV8.TBG.mLDLR ranging from 1.5 to 500×10¹¹ GC/kg and followed for reductions in plasma cholesterol (Kassim et al., 2010, PLoS One 5(10): e13424). The GC doses used in these research experiments (1.5 to 500×10¹¹) were based on quantitative PCR (qPCR) titer. Statistically significant reductions of plasma cholesterol of up to 30% were observed at day 21 at a dose of AAV8.TBG.mLDLR of 1.5×10¹¹ GC/kg, with greater reductions achieved in proportion to larger doses of vector (Kassim et al., 2010, PLoS One 5(10): e13424). Analyses of liver tissues harvested subsequent to metabolic correction revealed levels of mouse LDLR transgene and protein in proportion to the dose of vector. Thus, a dose-response correlation was observed.

7.4.2. Dose-Ranging Study of AAV8.TBG.hLDLR in DKO and LAHB Mice

Similar proof-of-concept studies in the DKO mouse were performed with a vector that contained the human LDL receptor (hLDLR) gene rather than the mouse LDLR gene. The results with the hLDLR vector were very similar to those observed with the mLDLR in that the dose of vector was proportional to expression of the transgene and deposition of vector genomes in liver (Kassim et al. 2013, Hum Gene Ther 24(1): 19-26). The major difference was in its efficacy—the human LDLR vector was less potent in this model. Reductions of cholesterol close to at least 30% were achieved at 5×10¹² GC/kg and 5×10¹¹ GC/kg, (doses based on qPCR titer) although statistical significance was achieved only at the higher dose.

The reduced efficacy observed was attributable to the diminished affinity of human LDLR for the mouse ApoB. To by-pass this problem, studies were repeated using the LAHB mouse model that expresses the human ApoB100 and, therefore, more authentically models the interaction of human apoB100 with human LDLR relevant to human studies. Male mice of both strains (DKO vs. LAHB) received a tail vein injection of one of three vector doses of AAV8.TBG.hLDLR (0.5×10¹¹ GC/kg, 1.5×10¹¹ GC/kg, and 5.0×10¹¹ GC/kg based on qPCR titer). Animals from each cohort were bled on day 0 (prior to vector administration), day 7, and day 21 and evaluation of serum cholesterol level was performed. The human LDLR was much more effective in the LAHB mouse as compared to mLDLR in the DKO mouse: a 30% reduction of serum cholesterol was achieved at a dose of 1.5×10¹¹ GC/kg, which is the same efficacy achieved with previous studies of the mouse LDLR construct in the DKO animals (Kassim et al. 2013, Hum Gene Ther 24(1): 19-26).

7.4.3. Non-Clinical Pharmacology/Toxicology Study of AAV8.TBG.mLDLR and AAV8.TBG.hLDLR in a Mouse Model of HoFH

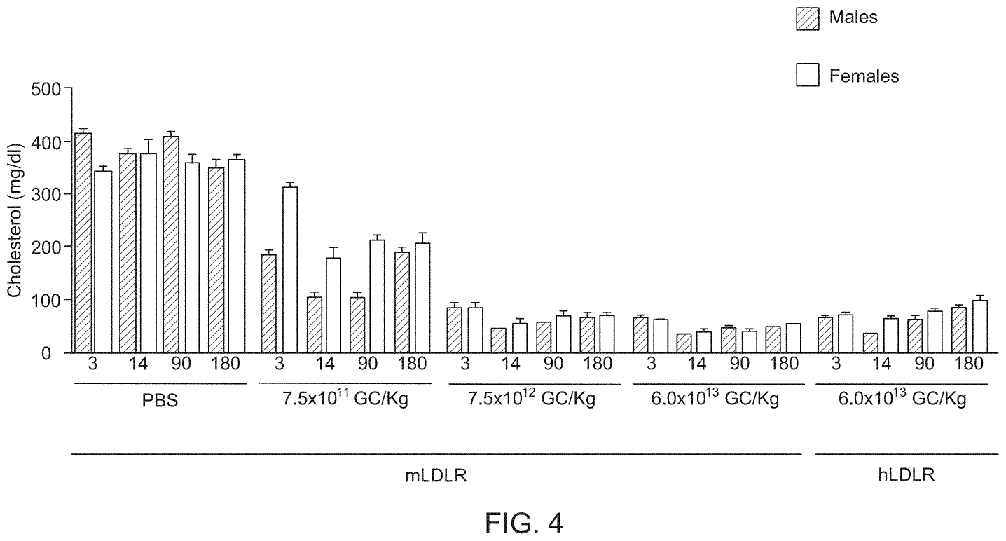

Male and female DKO mice (n=280, 140 male and 140 female) 6-22 weeks of age received a tail vein injection of one of three vector doses of AAV8.TBG.mLDLR (7.5×10¹¹ GC/kg, 7.5×10¹² GC/kg, 6.0×10¹³ GC/kg) or one dose of the intended gene therapy vector AAV8.TBG.hLDLR (6.0×10¹³ GC/kg). Animals were dosed based on genome copies (GC) per kilogram body weight using the oqPCR titration method, which is described herein at Section 8.4.1. An additional cohort of animals received PBS as a vehicle control. Animals from each cohort were sacrificed on day 3, day 14, day 90, and day 180 and blood was collected for evaluation of serum cholesterol levels (FIG. 4).

A rapid and significant reduction of cholesterol at all necropsy time points in all groups of treated mice was observed. This reduction appeared to be less in females than in males at low dose of vector at early time points, although this difference decreased with time and eventually there was no detectable difference between the sexes. Each group demonstrated a statistically significant reduction in serum cholesterol of at least 30% relative to PBS controls at the same necropsy time point. Therefore, the determination of the MED based on this study is ≤7.5×10¹¹ GC/kg.

7.4.4. Efficacy Study of AAV8.TBG.hLDLR in a Mouse Model of Homozygous Familial Hypercholesterolemia

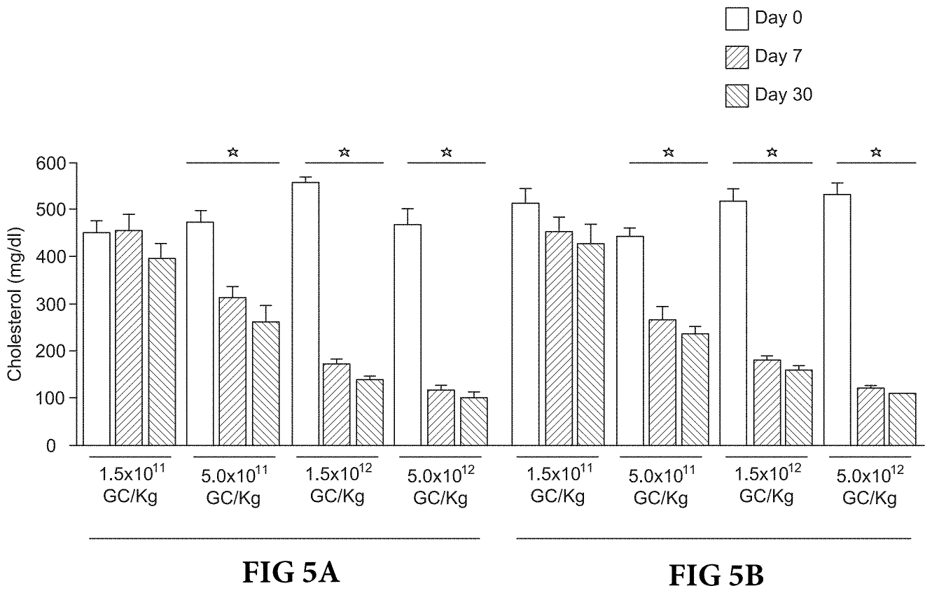

Male DKO mice (n=40) 12-16 weeks of age were administered IV with one of four doses (1.5×10¹¹ GC/kg, 5.0×10¹¹ GC/kg, 1.5×10¹² GC/kg, 5.0×10¹² GC/kg) of AAV8.TBG.hLDLR (doses based on the oqPCR titration method). Animals were bled on day 0 (prior to vector administration), day 7, and day 30 and evaluation of serum cholesterol (FIG. 5). A rapid and significant reduction of cholesterol was observed on days 7 and 30 in groups of mice treated with ≥5.0×10¹¹ GC/kg. The determination of the MED based on this study is between 1.5×10¹¹ GC/kg and 5.0×10¹¹ GC/kg.

7.5. Effects of AAV8.TBG.rhLDLR in LDLR+/− Rhesus Macaques on a High-Fat Diet

Studies designed to evaluate AAV8-LDLR gene transfer in the FH macaque were conducted. Following administration of 10¹³ GC/kg of AAV8.TBG.rhAFP (a control vector; dose based on qPCR titration method) into either fat-fed or chow fed wild type rhesus macaques, no elevations in aspartate aminotransferase (AST) or alanine aminotransferase (ALT) values were seen. This suggests that AAV8 capsid itself is not responsible for triggering an inflammatory or injurious hepatic process.

7.6. Pilot Biodistribution Study of AAV8.TBG.hLDLR in a Mouse Model of HoFH

In order to assess the safety and pharmacodynamics properties of gene therapy for HoFH, pilot biodistribution (BD) studies were conducted in DKO mice. These studies examined vector distribution and persistence in five female DKO mice systemically administered 5×10¹² GC/kg (dose based on qPCR titration method) of AAV8.TBG.hLDLR vector via one of two routes: 1) IV injection into the tail vein or 2) intra-portal injection. At two different time points (day 3 and day 28), a panel of tissues was harvested and total cellular DNA was extracted from harvested tissues. In these pilot studies, both the IV and intra-portal routes resulted in a comparable BD profile, supporting the rationale to infuse the gene therapy vector in patients and animals via peripheral vein.

7.7. Toxicology

In order to assess the potential toxicity of gene therapy for HoFH, pharmacology/toxicology studies were conducted in DKO mice (a mouse model of HoFH), and wild type and LDLR+/− rhesus macaques. The studies include an examination of the role of LDLR transgene expression in vector associated toxicity in chow-fed wild type and LDLR+/− Rhesus Macaques, a pharmacology/toxicology study of AAV8.TBG.mLDLR and AAV8.TBG.hLDLR in a mouse model of HoFH, and an examination of the non-clinical biodistribution of AAV8.TGB.hLDLR in a mouse model of HoFH. These studies are described in detail below.

7.8. Non-Clinical Study Examining the Role of LDLR Transgene Expression in Vector Associated Toxicity in Chow-Fed Wild Type and LDLR+/− Rhesus Macaques

Four wild type and four LDLR+/− rhesus macaques were administered IV with 1.25×10¹³ GC/kg of AAV8.TBG.hLDLR (dose based on oqPCR titration method), Non human primates (NHPs) were monitored for up to one year post-vector administration. Four animals (two wild type and two LDLR+/−) were necropsied at day 28 post-vector administration to assess acute vector-associated toxicity and vector distribution and four animals (two wild type and two LDLR+/−) were necropsied at day 364/365 post-vector administration to assess long-term vector-associated pathology and vector distribution. Each cohort of wild type and LDLR+/− macaques had two males and two females.

The animals tolerated the infusion of vector well without long-term or short-term clinical sequelae. Biodistribution studies demonstrated high level and stable targeting of liver with far less, but still detectable, extrahepatic distribution, which declined over time. These data suggested that the target organ for efficacy, the liver, is also the most likely source of potential toxicity. A detailed review of tissues harvested at necropsy performed 28 and 364/365 days post-vector administration revealed some minimal to mild findings in liver and some evidence of atherosclerosis in the LDLR+/− macaques. The nature of the liver pathology and the fact that similar pathology was observed in one of the two untreated wild type animals suggested to the pathologist that they were unrelated to the test article.

One animal had persistent elevations in alanine aminotransferase (ALT) prior to vector administration, which continued after vector administration at levels that ranged from 58 to 169 U/L. The remaining animals demonstrated either no elevations in transaminases or only transient and low level increases in aspartate aminotransferase (AST) and ALT, never exceeding 103 U/L. The most consistent abnormalities were found after vector injection, suggesting they were related to the test article. Activation of T cells to human LDLR or to AAV8 capsid was assessed for correlation with AST/ALT increases. FIG. 6 presents the AAV capsid ELISPOT data and serum AST levels in three selected animals that demonstrated relevant findings. Only one animal showed a correlation in which an increase in AST to 103 U/L corresponded to the appearance of T cells against capsid (FIG. 6, animal 090-0263); the capsid T cell response persisted while the AST returned immediately to normal range.

Analysis of tissue-derived T cells for presence of capsid and transgene-specific T cells showed that liver derived T cells became responsive to capsid from both genotypes (wild type and LDLR+/−) by the late time point while T cells to human LDLR were detected in the LDLR+/− animals at this late time point. This suggests that PBMCs are not reflective of the T cell compartment in the target tissue. Liver tissue harvested at days 28 and at 364/365 was analyzed for expression of the transgene by RT-PCR and did appear to be affected by the abnormalities in clinical pathology or the appearance of T cells.

Neither the wild type nor LDLR+/− animals developed hypercholesterolemia on chow diet. Dose-Limiting Toxicities (DLTs) were not observed at a dose of 1.25×10¹³ GC/kg (based on oqPCR), implying that the maximal tolerated dose (MTD) would be equal to or greater than this dose. Test article related elevations in transaminases were observed, which were low and transient but nevertheless present. Accordingly, the no-observed-adverse-effect-level (NOAEL) is less than the single high dose evaluated in Example 1 herein.

7.9. Non-Clinical Pharmacology/Toxicology Study of AAV8.TBG.mLDLR and AAV8.TBG.hLDLR in a Mouse Model of HoFH

This study was conducted in the DKO mice because using this strain would allow, 1) evaluation of proof-of-concept efficacy in parallel with toxicity, and 2) evaluation of vector-associated toxicity in the setting of any pathology associated with the defect in LDLR and the associated dyslipidemia and its sequelae, such as steatosis.

The study was designed to test AAV8.TBG.hLDLR at the highest dose, which is 8-fold higher than the highest dose for administration to human subjects with HoFH, as set forth in Example 1. A version of the vector that expresses the murine LDLR was tested at this high dose, as well as two lower doses, to provide an assessment of the effect of dose on toxicity parameters, as well as reduction in cholesterol. The dose-response experiment was performed with the vector expressing murine LDLR to be more reflective of the toxicity and efficacy that would be observed in humans using the human LDLR vector.

In this study, male and female DKO mice aged 6-22 weeks were administered with one of the doses of AAV8.TBG.mLDLR (7.5×10¹¹ GC/kg, 7.5×10¹² GC/kg and 6.0×10¹³ GC/kg) or 6.0×10¹³ GC/kg of the vector (AAV8.TBG.hLDLR) (doses based on the oqPCR titration method). Animals were necropsied at day 3, day 14, day 90, and day 180 post-vector administration; these times were selected to capture the vector expression profile of the test article as well as acute and chronic toxicity. Efficacy of transgene expression was monitored by measurement of serum cholesterol levels. Animals were evaluated for comprehensive clinical pathology, immune reactions to the vector (cytokines, NAbs to AAV8 capsid, and T cell responses against both capsid and transgene), and tissues were harvested for a comprehensive histopathological examination at the time of necropsy.

The key toxicology findings from this study are as follows:
No clinical sequelae were observed in the treated groups
Clinical pathology:
   Transaminases: Abnormalities were limited to elevations of the liver function tests AST and ALT that ranged from 1-4×ULN and were primarily found at day 90 of all doses of murine LDLR vector. There was no elevation of transaminases in the group administered high dose human LDLR vector, except for <2×ULN of ALT in a few male animals. The abnormalities associated with the mouse vector were mild and not dose-dependent and, therefore, were not believed to be related to vector. There were essentially no findings associated with the high dose human vector. There was no evidence of treatment related toxicity based on these findings, meaning that the no adverse effect level (NOAEL) based on these criteria is 6.0×10¹³ GC/kg.
Pathology: There were no gross pathology findings. Histopathology was limited to minimal or mild findings in liver as follows:
   Animals administered with PBS had evidence of minimal and/or mild abnormalities according to all criteria evaluated. In assessing treatment related pathology we focused on any finding categorized as mild that was above that found in PBS injected animals.
   Mild bile duct hyperplasia and sinusoidal cell hyperplasia was observed in high dose female mice administered the mouse and human LDLR vectors. This could represent vector related effects observed only at the high dose.
   Centrilobular hypertrophy was mild, only in males and not at high doses of vector arguing that it not vector related.
   Minimal necrosis was found in 1/7 males and 3/7 females at day 180 in the high dose human LDLR vector.
   Based on the finding of mild bile duct and sinusoidal hyperplasia at the high dose of vector, and a few examples of minimal necrosis in the high dose human LDLR vector, that the NOAEL based on these criteria is between 7.5×10¹² GC/kg and 6.0×10¹³ GC/kg.
Other findings: The animals developed an increase in NAbs to AAV8 and evidence of very low T cell response based on an IFN-γ ELISPOT to capsid and LDLR following administration of the high dose of the human LDLR vector. There was little evidence of an acute inflammatory response based on analysis of serum 3 and 14 days after vector; a few cytokines did show modest and transient elevations although there was no increase in IL6.

One notable finding was that toxicity was not worse in DKO mice treated with the mouse LDLR vector than with the human LDLR vector, which could have been the case if the human LDLR was more immunogenic in terms of T cells than the mouse transgene. ELISPOT studies did show some activation of LDLR-specific T cells in mice administered with the high dose vector expressing the human transgene, although they were low and in a limited number of animals supporting the toxicity data, which suggested this mechanism of host response would unlikely contribute to safety concerns.

In conclusion, there were no dose-limited toxicities, meaning the maximally tolerated dose was higher than the highest dose tested which was 6.0×10¹³ GC/kg. Based on mild and reversible findings in liver pathology at the highest dose, the NOAEL is somewhere between 6.0×10¹³ GC/kg, where in liver mild reversible pathology was observed, down to 7.5×10¹² GC/kg, where there was no clear indication of vector related findings.

7.10. Non-Clinical Biodistribution of AAV8.TGB.hLDLR in a Mouse Model of HoFH

Male and female DKO mice 6-22 weeks of age were administered IV with 7.5×10¹² GC/kg (dose measured by oqPCR titration method) of AAV8.TBG.hLDLR, the highest dose for treating human subjects in Example 1 f. Animals were necropsied for biodistribution assessment on day 3, day 14, day 90, and day 180 post-vector administration. In addition to blood, 20 organs were harvested. The distribution of vector genomes in organs was assessed by quantitative, sensitive PCR analysis of total genomic DNA harvested. One sample of each tissue included a spike of control DNA, including a known amount of the vector sequences, in order to assess the adequacy of the PCR assay reaction.

The vector GC number in liver was substantially higher in liver than in other organs/tissues, which is consistent with the high hepatotropic properties of the AAV8 capsid. For example, vector genome copies in the liver were at least 100-fold greater than that found in any other tissue at day 90. There was no significant difference between male or female mice at the first three time points. GC number decreased over time in the liver until day 90, where it then stabilized. A similar trend of decline was observed in all tissues but the decline in vector copy number was more rapid in tissues with higher cell turnover rate. Low but detectable levels of vector genome copies were present in the gonads of both genders and the brain.

The biodistribution of AAV8.TBG.hLDLR in DKO mice was consistent with published results with AAV8. Liver is the target primary target of gene transfer following IV infusion and genome copies in liver do not decline significantly over time. Other organs are targeted for vector delivery, although the levels of gene transfer in these non-hepatic tissues are substantially lower and decline over time. Therefore, the data presented here suggest that the primary organ system to be evaluated is the liver.

7.11. Conclusions from Non-Clinical Safety Studies

The rhesus macaque and DKO mouse studies confirmed that high dose vector is associated with low level, transient, and asymptomatic liver pathology evident by transient elevations in transaminases in NHPs, and in mice by transient appearance of mild bile duct and sinusoidal hypertrophy. No other toxicity felt to be due to the vector was observed.

There were no DLTs observed at doses as high as 1.25×10¹³ GC/kg in macaques and 6×10¹³ GC/kg in DKO mice. Determination of the NOAEL focus primarily on liver toxicity as reflected in elevations in transaminases in macaques and histopathology in DKO mice. This translated to an NOAEL of less than 1.25×10¹³ GC/kg in macaques and less than 6×10¹³ GC/kg but greater than 7.5×10¹² GC/kg in DKO mice. The doses were based on the oqPCR titration method.

7.12. Overall Assessment of Non-Clinical Data to Support Human Treatment

The key findings that emerged from the pharmacology and toxicology studies that have informed the dose selection and design for the clinical study, are the following:
Minimal Effective Dose (MED): The MED was defined in nonclinical studies as a GC/kg dose that resulted in a 30% reduction in serum cholesterol. Two IND-enabling nonclinical studies established the MED to be between 1.5 to 5.0×10¹¹ GC/kg. The mouse pharmacology/toxicology study demonstrated a statistically significant reduction in serum cholesterol of at least 30% relative to PBS controls, allowing estimation of a MED ≤7.5×10¹¹ GC/kg. The observed dose-response relationship allowed determination of the MED to be between 1.5 to 5.0×10¹¹ GC/kg as determined by oqPCR.
Maximum Tolerated Dose (MTD): The MTD was defined in nonclinical studies as the GC/kg dose that did not result in a dose limiting toxicity (DLT). DLTs were not observed in the toxicology studies at the highest doses tested, which were 6.0×10¹³ GC/kg in DKO mice and 1.25×10¹³ GC/kg in macaques as determined by oqPCR. Our results suggested that the actual MTD is higher than these doses.

In mice given AAV8.TBG.hLDLR at a dose of 6.0×10¹³ GC/kg, there were no adverse effects seen following 3, 14, 90 or 180 days of treatment. In monkeys and mice given AAV8.TBG.hLDLR, occasional increases in transaminases were reported in both monkeys and mice. In mice, minimal necrosis in the liver was observed in AAV8.TBG.hLDLR treated mice on Day 180 only. However, it was not observed on Day 90 or in any animal given the murine transgene product likely suggesting it may have been associated with an immune response to the human transgene product. Whilst no clear adverse effects were observed in mice or monkeys given AAV8.TBG.hLDLR, the minimal elevations in ALT and AST are in accordance with clinical data describing the potential for AAVs to elicit hepatic effects.
No Observed Adverse Event Level (NOAEL): This was determined to be 7.5×10¹² GC/kg in the DKO mice. This was based on minimal to mild histopathologic findings, predominantly in the liver (bile duct and sinusoidal hyperplasia, minimal necrosis), observed at higher doses of the human LDLR (hLDLR) transgene. Only one dose was tested in macaques; however the toxicity at 1.25×10¹³ GC/kg was mild, including transient and low level increases in AST and ALT, suggesting the true NOAEL would be achieved at a dose lower than the dose tested.

Based on these data, three single-dose cohorts were proposed, 2.5×10¹² GC/kg, 7.5×10¹² GC/kg, and 2.5×10¹³ GC/kg (doses based on the oqPCR method). These doses represent half-log, stepwise increases that could inform a dose-response and that represent a dose range that is supported by the non-clinical testing. The introduction of prophylactic corticosteroids in the clinical protocol is anticipated to improve the safety of product administration by attenuating or preventing immune mediated hepatocyte injury. T

### 8. Example 3: Manufacture of AAV8.TBG.hLDLR

The AAV8.TBG.hLDLR vector consists of the AAV vector active ingredient and a formulation buffer. The external AAV vector component is a serotype 8, T=1 icosahedral capsid consisting of 60 copies of three AAV viral proteins, VP1, VP2, and VP3, at a ratio of 1:1:18. The capsid contains a single-stranded DNA recombinant AAV (rAAV) vector genome (FIG. 7). The genome contains a human low density lipoprotein receptor (LDLR) transgene flanked by the two AAV inverted terminal repeats (ITRs). An enhancer, promoter, intron, human LDLR coding sequence and polyadenylation (polyA) signal comprise the human LDLR transgene. The ITRs are the genetic elements responsible for the replication and packaging of the genome during vector production and are the only viral cis elements required to generate rAAV. Expression of the human LDLR coding sequence is driven from the hepatocyte-specific thyroxine-binding globulin (TBG) promoter. Two copies of the alpha 1 microglobulin/bikunin enhancer element precede the TBG promoter to stimulate promoter activity. A chimeric intron is present to further enhance expression and a rabbit beta globin polyA signal is included to mediate termination of human LDLR mRNA transcripts. The vector is supplied as a suspension of AAV8. TBG.hLDLR vector in formulation buffer. The formulation buffer is 180 mM NaCl, 10 mM sodium phosphate, 0.001% Poloxamer 188, pH 7.3.

Details of the vector manufacturing and characterization of the vectors, are described in the sections below.

8.1. Plasmids Used to Produce AAV8.TBG.hLDLR

The plasmids used for production of AAV8.TBG.hLDLR are as follows:

8.1.1 Cis Plasmid (Vector Genome Expression Construct):

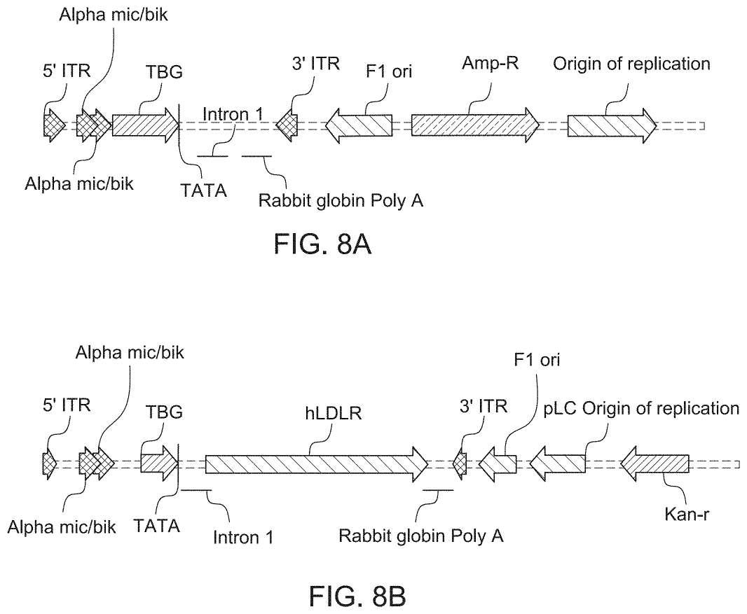

pENN.AAV.TBG.hLDLR.RBG.KanR containing the human LDLR expression cassette (FIG. 8). This plasmid encodes the rAAV vector genome. The polyA signal for the expression cassette is from the rabbit 0 globin gene. Two copies of the alpha 1 microglobulin/bikunin enhancer element precede the TBG promoter.

To generate the cis plasmid used for production of AAV8.TBG.hLDLR, the human LDLR cDNA was cloned into an AAV2 ITR-containing construct, pENN.AAV.TBG.PI to create pENN.AAV.TBG.hLDLR.RBG. The plasmid backbone in pENN.AAV.TBG.PI was originally from, pZac2.1, a pKSS-based plasmid. The ampicillin resistance gene in pENN.AAV.TBG.hLDLR.RBG was excised and replaced with the kanamycin gene to create pENN.AAV.TBG.hLDLR.RBG.KanR. Expression of the human LDLR cDNA is driven from the TBG promoter with a chimeric intron (Promega Corporation, Madison, Wis.). The polyA signal for the expression cassette is from the rabbit 0 globin gene. Two copies of the alpha 1 microglobulin/bikunin enhancer element precede the TBG promoter.

Description of the Sequence Elements

1. Inverted terminal repeats (ITR): AAV ITRs (GenBank #NC001401) are sequences that are identical on both ends, but found in opposite orientation. The AAV2 ITR sequences function as both the origin of vector DNA replication and the packaging signal for the vector genome, when AAV and adenovirus (ad) helper functions are provided in trans. As such, the ITR sequences represent the only cis acting sequences required for vector genome replication and packaging.
2. Human alpha 1 microglobulin bikunin enhancer (2 copies; 0.1 Kb); Genbank #X67082) This liver specific enhancer element serves to lend liver-specificity and enhance expression from the TBG promoter.
3. Human thyroxine-binding globulin (TBG) promoter (0.46 Kb; Gen bank #L13470) This hepatocyte-specific promoter drives the expression of the human LDLR coding sequence
4. Human LDLR cDNA (2.58 Kb; Genbank #NM000527, complete CDS). The human LDLR cDNA encodes a low density lipoprotein receptor of 860 amino acids with a predicted molecular weight of 95 kD and an apparent molecular weight of 130 kD by SDS-PAGE.
5. Chimeric intron (0.13 Kb; Genbank #U47121; Promega Corporation, Madison, Wis.) The chimeric intron consists of a 5′-donor site from the first intron of the human β-globin gene and the branch and 3′-acceptor site from the intron located between the leader and body of an immunoglobulin gene heavy chain variable region. The presence of an intron in an expression cassette has been shown to facilitate the transport of mRNA from the nucleus to the cytoplasm, thus enhancing the accumulation of the steady level of mRNA for translation. This is a common feature in gene vectors intended to mediate increased levels of gene expression.
6. Rabbit beta-globin polyadenylation signal: (0.13 Kb; GenBank #V00882.1) The rabbit beta-globin polyadenylation signal provides cis sequences for efficient polyadenylation of the antibody mRNA. This element functions as a signal for transcriptional termination, a specific cleavage event at the 3′ end of the nascent transcript followed by addition of a long polyadenyl tail.

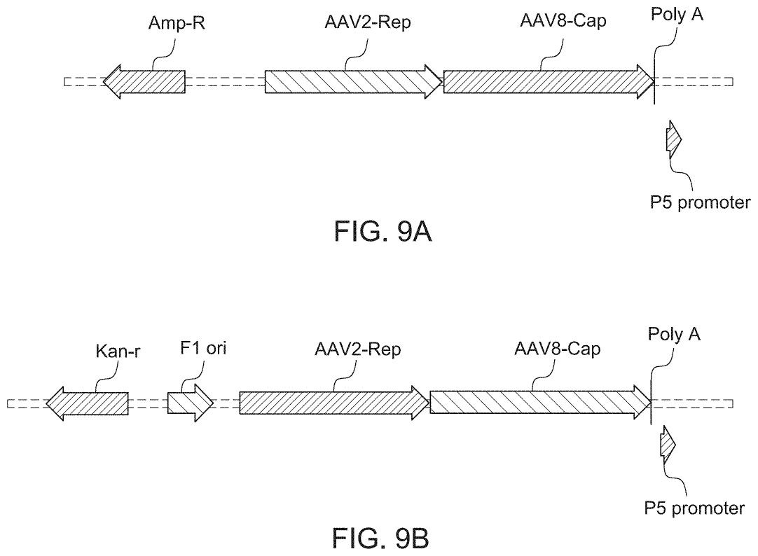

8.1.2 Trans Plasmid (Packaging Construct): pAAV2/8(Kan), Containing the AAV2 Rep Gene and AAV8 Cap Gene (FIG. 9).

The AAV8 trans plasmid pAAV2/8(Kan) expresses the AAV2 replicase (rep) gene and the AAV8 capsid (cap) gene encoding virion proteins, VP1, VP2 and VP3. The AAV8 capsid gene sequences were originally isolated from heart DNA of a rhesus monkey (GenBank accession AFS13852). To create the chimeric packaging constructs, plasmid p5E18, containing AAV2 rep and cap genes, was digested with XbaI and XhoI to remove the AAV2 cap gene. The AAV2 cap gene was then replaced with a 2.27 Kb SpeI/XhoI PCR fragment of the AAV8 cap gene to create plasmid p5E18VD2/8 (FIG. 9a). The AAV p5 promoter, which normally drives rep expression is relocated in this construct from the 5′ end of rep gene to the 3′ end of the cap gene. This arrangement serves to down-regulate expression of rep in order to increase vector yields. The plasmid backbone in p5E18 is from pBluescript KS. As a final step, the ampicillin resistance gene was replaced by the kanamycin resistance gene to create pAAV2/8(Kan) (FIG. 9B). The entire pAAV2/8(Kan) trans plasmid has been verified by direct sequencing.

8.1.3 Adenovirus Helper Plasmid: pAdΔF6(Kan)

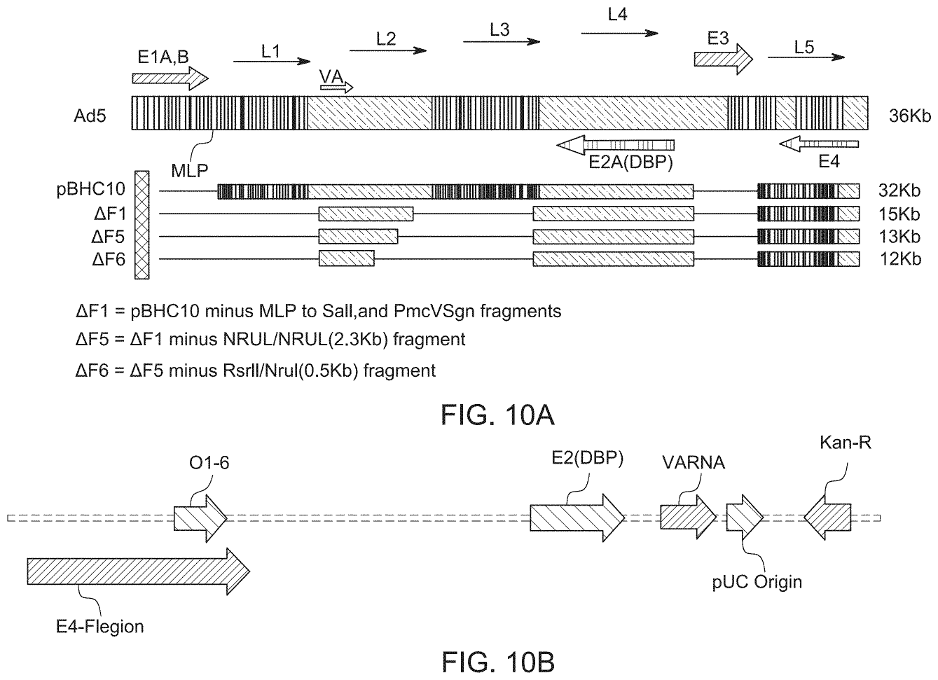

Plasmid pAdΔF6(Kan) is 15.7 Kb in size and contains regions of the adenoviral genome that are important for AAV replication, namely E2A, E4, and VA RNA. pAdΔF6(Kan) does not encode any additional adenoviral replication or structural genes and does not contain cis elements, such as the adenoviral ITRs, that are necessary for replication, therefore, no infectious adenovirus is expected to be generated. Adenoviral E1 essential gene functions are supplied by the HEK293 cells in which the rAAV vectors are produced. pAdΔF6(Kan) was derived from an E1, E3 deleted molecular clone of Ad5 (pBHG10, a pBR322 based plasmid). Deletions were introduced in the Ad5 DNA to remove unnecessary adenoviral coding regions and reduce the amount of adenoviral DNA from 32 Kb to 12 Kb in the resulting ad-helper plasmid. Finally, the ampicillin resistance gene was replaced by the kanamycin resistance gene to create pAdΔF6(Kan) (FIG. 10). DNA plasmid sequencing was performed by Qiagen Sequencing Services, Germany and revealed 100% homology between the reference sequence for pAdDeltaF6(Kan) and the following adenoviral elements: p1707FH-Q: E4 ORF6 3.69-2.81 Kb; E2A DNA binding protein 11.8-10.2 Kb; VA RNA region 12.4-13.4 Kb.

Each of the cis, trans and ad-helper plasmids described above contains a kanamycin-resistance cassette, therefore, β-lactam antibiotics are not used in their production.

8.1.4 Plasmid Manufacturing

All plasmids used for the production of vectors were produced by Puresyn Inc. (Malvern, Pa.). All growth media used in the process is animal free. All components used in the process, including fermentation flasks, containers, membranes, resin, columns, tubing, and any component that comes into contact with the plasmid, are dedicated to a single plasmid and are certified BSE-free. There are no shared components and disposables are used when appropriate.

8.2. Cell Banks

AAV8.TBG.hLDLR vector was produced from a HEK293 working cell bank which was derived from a fully characterized master cell bank. The manufacturing and testing details of both cell banks appears below.

8.2.1 HEK293 Master Cell Bank

HEK293 Master Cell Bank (MCB) is a derivative of primary human embryonic kidney cells (HEK) 293. The HEK293 cell line is a permanent line transformed by sheared human adenovirus type 5 (Ad5) DNA (Graham et al., 1977, Journal of General Virology 36(1): 59-72). The HEK293 MCB has been tested extensively for microbial and viral contamination. The HEK293 MCB is currently stored in liquid nitrogen. Additional testing was performed on the HEK293 MCB to demonstrate the absence of specific pathogens of human, simian, bovine, and porcine origin. The human origin of the HEK293 MCB was demonstrated by isoenzyme analysis.

Tumorigenicity testing was also performed on the HEK293 MCB by evaluating tumor formation in nude (nu/nu) athymic mice following subcutaneous injection of the cell suspension. In this study, fibrosarcoma was diagnosed at the injection site in ten of ten positive control mice and carcinoma was diagnosed at the injection site in ten of ten test article mice. No neoplasms were diagnosed in any of the negative control mice. The HEK293 MCB L/N 3006-105679 was also tested for the presence of Porcine Circovirus (PCV) Types 1 and 2. The MCB was found negative for PCV types 1 and 2.

8.2.2 HEK293 Working Cell Bank

The HEK293 Working Cell Bank (WCB) was manufactured using New Zealand sourced Fetal Bovine Serum, FBS (Hyclone PN SH30406.02) certified for suitability in accordance with the European Pharmacopea monograph. The HEK293 WCB was established using one vial (1 mL) of the MCB as seed material. Characterization tests were performed and the test results are listed in Table 4.1.

TABLE 4.1
Characterization of HEK293 WCB.

| Test | Method | Study Number | Result |
|---|---|---|---|
| Test for the | In vivo | BioReliance | No |
| presence of agar- | | AD61FS.102063GMP.BSV | myco- |
| cultivable and non- | | | plasma |
| agar cultivable | | | detected |
| mycoplasma USP, | | | |
| EP, 1993 PTC | | | |
| Qualification of | In vivo | BioReliance | No |
| the test for agar- | | AD61FS.102062GMP.BSV | Myco- |
| cultivable and non- | | | plasma- |
| agar cultivable | | | stasis |
| mycoplasma USP, | | | observed |
| EP, 1993 PTC/JP | | | |
| Isolator sterility | Direct | BioReliance | No |
| testing, USP <71>, | inocu- | AD61FS.510120GMP.BSV | bacterial |
| 21 CFR 610.12 | lation | | or |
| | | | fungal |
| | | | growth |
| Test for | In vivo | BioReliance | Negative |
| presence of | | AD61FS.005002GMP.BSV | |
| inapparent | | | |
| viruses | | | |
| 28-day assay for | In vitro | BioReliance | Negative |
| the presence | | AD61FS.003800.BSV | |
| of viral | | | |
| contaminants | | | |
| Cell culture | Iso- | BioReliance | Human |
| identification and | enzyme | AD61FS.380801.BSV | |
| characterization | analysis | | |

8.3. Vector Manufacturing

General descriptions of the vector manufacturing processes are given below and are also reflected in a flow diagram in FIG. 11.

8.3.1 Vector Generation Process (Upstream Process)

8.3.1.1 Initiation of HEK293 WCB Cell Culture into a T-Flask (75 cm²)

One vial of HEK293 cells from the WCB containing 10⁷ cells in 1 mL is thawed at 37° C. and seeded in a 75 cm² tissue culture flask containing DMEM High Glucose supplemented with 10% fetal bovine serum (DMEM HG/10% FBS). The cells are then placed in a 37° C./5% CO2 incubator, and grown to ˜70% confluence with daily direct visual and microscopic inspection to assess cell growth. These cells are designated Passage 1 and are passaged to generate a cell seed train for vector biosynthesis for up to ˜10 weeks as described below. The passage number is recorded at each passage and the cells are discontinued after passage 20. If additional cells are required for vector biosynthesis, a new HEK293 cell seed train is initiated from another vial of the HEK293 WCB.

8.3.1.2 Passage of Cells into ˜2 T-Flasks (225 cm²)

When the HEK293 cells growing in the T75 flask are ˜70% confluent, the cells are detached from the surface of the flask using recombinant trypsin (TrypLE) and seeded in two T225 flasks containing DMEM HG/10% FBS. Cells are placed in the incubator and grown to ˜70% confluence. Cells are monitored for cell growth, absence of contamination, and consistency by visual inspection and using a microscope.

8.3.1.3 Passage of Cells into ˜10 T-Flasks (225 cm²)

When the HEK293 cells growing in the two T225 flask are ˜70% confluent, the cells are detached using recombinant trypsin (TrypLE), and seeded at a density of ˜3×10⁶ cells per flask in ten 225 cm2 T-flasks containing DMEM HG/10% FBS. Cells are placed in a 37° C./5% CO₂ incubator and grown to ˜70% confluence. Cells are monitored for cell growth, absence of contamination, and consistency by direct visual inspection and using a microscope. Cells are maintained by serial passaging in T225 flasks to maintain the cell seed train and to provide cells for expansion to support manufacture of subsequent vector batches.

8.3.1.4 Passage of Cells into ˜10 Roller Bottles

When the HEK293 cells growing in ten T225 flasks are ˜70% confluent, the cells are detached using recombinant trypsin (TrypLE), counted and seeded in 850 cm² roller bottles (RB) containing DMEM HG/10% FBS. The RBs are then placed in the RB incubator and the cells grown to ˜70% confluence. RBs are monitored for cell growth, absence of contamination, and consistency by direct visual inspection and using a microscope.

8.3.1.5 Passage of Cells into ˜100 Roller Bottles

When the HEK293 cells growing in RBs prepared as described in the previous process step are ˜70% confluent, they are detached using recombinant trypsin (TrypLE), counted and seeded in 100 RBs containing DMEM/10% FBS. The RBs are then placed in the RB incubator (37° C., 5% CO₂) and grown to ˜70% confluence. Cells are monitored for cell growth, absence of contamination, and consistency by direct visual inspection and using a microscope.

8.3.1.6 Transfection of Cells with Plasmid DNA

When the HEK293 cells growing in 100 RBs are ˜70% confluent, the cells are transfected with each of the three plasmids: the AAV serotype-specific packaging (trans) plasmid, the ad-helper plasmid, and vector cis plasmid containing the expression cassette for the human LDLR gene flanked by AAV inverted terminal repeats (ITRs). Transfection is carried out using the calcium phosphate method (For plasmid details, see Section 4.1.1). The RBs are placed in the RB incubator (37° C., 5% CO₂) overnight.

8.3.1.7. Medium Exchange to Serum Free Medium

After overnight incubation of 100 RBs following transfection, the DMEM/10% FBS culture medium containing transfection reagents is removed from each RB by aspiration and replaced with DMEM-HG (without FBS). The RBs are returned to the RB incubator and incubated at 37° C., 5% CO₂ until harvested.

8.3.1.8. Vector Harvest

RBs are removed from the incubator and examined for evidence of transfection (transfection-induced changes in cell morphology, detachment of the cell monolayer) and for any evidence of contamination. Cells are detached from the RB surface by agitation of each RB, and then harvested by decanting into a sterile disposable funnel connected to a BioProcess Container (BPC). The combined harvest material in the BPC is labeled ‘Product Intermediate: Crude Cell Harvest’ and samples are taken for (1) in-process bioburden testing and (2) bioburden, mycoplasma, and adventitious agents product release testing. The Product Intermediate batch labeled as Crude Cell Harvest (CH) is stored at 2-8° C. until further processed.

8.3.2 Vector Purification Process (Downstream Process)

While a common, ‘platform’ purification process is used for all of the AAV serotypes (i.e. incorporating the same series and order of steps), each serotype requires unique conditions for the chromatography step, a requirement that also impacts some details (buffer composition and pH) of the steps used to prepare the clarified cell lysate applied to the chromatography resin.

8.3.2.1 AAV8 Vector Harvest Concentration and Diafiltration by TFF

The BPC containing Crude CH is connected to the inlet of the sanitized reservoir of a hollow fiber (100k MW cut-off) TFF apparatus equilibrated with phosphate-buffered saline. The Crude CH is applied to the TFF apparatus using a peristaltic pump and concentrated to 1-2 L. The vector is retained (retentate) while small molecular weight moieties and buffer pass through the TFF filter pore and are discarded. The harvest is then diafiltered using the AAV8 diafiltration buffer. Following diafiltration, the concentrated vector is recovered into a 5 L BPC. The material is labeled ‘Product Intermediate: Post Harvest TFF’, and a sample taken for in-process bioburden testing. The concentrated harvest is further processed immediately or stored at 2-8 C until further processing.

8.3.2.2 Microfluidization and Nuclease Digestion of Harvest

The concentrated and diafiltered harvest is subjected to shear that breaks open intact HEK293 cells using a microfluidizer. The microfluidizer is sanitized with 1N NaOH for a minimum of 1 h after each use, stored in 20% ethyl alcohol until the next run, and rinsed with WFI prior to each use. The crude vector contained in the BPC is attached to the sanitized inlet port of the microfluidizer, and a sterile empty BPC is attached to the outlet port. Using air pressure generated by the microfluidizer, vector-containing cells are passed through the microfluidizer interaction chamber (a convoluted 300 μm diameter pathway) to lyse cells and release vector. The microfluidization process is repeated to ensure complete lysis of cells and high recovery of vector. Following the repeat passage of the product intermediate through the microfluidizer, the flowpath is rinsed with ˜500 mL of AAV8 Benzonase Buffer. The 5 L BPC containing microfluidized vector is detached from the outlet port of the microfluidizer. The material is labeled ‘Product Intermediate: Final Microfluidized’, and samples are taken for in-process bioburden testing. The microfluidized product intermediate is further processed immediately or stored at 2-8° C. until further processing. Nucleic acid impurities are removed from AAV8 particles by additional of 100 U/mL Benzonase®. The contents of the BPC are mixed and incubated at room temperature for at least 1 hour. Nuclease digested product intermediate is processed further.

8.3.2.3 Filtration of Microfluidized Intermediate

The BPC containing microfluidized and digested product intermediate is connected to a cartridge filter with a gradient pore size starting at 3 μm going down to 0.45 μm. The filter is conditioned with AAV Benzonase Buffer. Using the peristaltic pump, the microfluidized product intermediate is passed through the cartridge filter and collected in the BPC connected to the filter outlet port. Sterile AAV8 Benzonase Buffer is pumped through the filter cartridge to rinse the filter. The filtered product intermediate is then connected to a 0.2 μm final pore size capsule filter conditioned with AAV8 Benzonase Buffer. Using the peristaltic pump, the filtered intermediate is passed through the cartridge filter and collected in the BPC connected to the filter outlet port. A volume of sterile AAV8 Benzonase Buffer is pumped through the filter cartridge to rinse the filter. The material is labeled ‘Product Intermediate: Post MF 0.2 μm Filtered’, and samples taken for in-process bioburden testing. The material is stored overnight at 2-8° C. until further processing. An additional filtration step may be performed on the day of chromatography prior to application of the clarified cell lysate to the chromatography column.

8.3.2.4 Purification by Anion-Exchange Chromatography

The 0.2 μm filtered Product Intermediate is adjusted for NaCl concentration by adding Dilution Buffer AAV8. The cell lysate containing vector is next purified by ion exchange chromatography using ion exchange resin. The GE Healthcare AKTA Pilot chromatography system is fitted with a BPG column containing approximately 1 L resin bed volume. The column is packed using continuous flow conditions and meets established asymmetry specifications. The system is sanitized according to the established procedure and is stored in 20% ethyl alcohol until the next run. Immediately prior to use, the system is equilibrated with sterile AAV8 Wash Buffer. Using aseptic techniques and sterile materials and components, the BPC containing clarified cell lysate is connected to the sanitized sample inlet port, and BPC's containing bioprocessing buffers listed below are connected to sanitized inlet ports on the AKTA Pilot. All connections during the chromatography procedure are performed aseptically. The clarified cell lysate is applied to the column and rinsed using AAV8 Wash Buffer. Under these conditions, vector is bound to the column, and impurities are rinsed from the resin. AAV8 particles are eluted from the column by application of AAV8 Elution buffer and collected into a sterile plastic bottle. The material is labeled ‘Product Intermediate and samples are taken for in-process bioburden testing. The material is further processed immediately.

8.3.2.5 Purification by CsCl Gradient Ultracentrifugation

The AAV8 particles purified by anion exchange column chromatography as described above contain empty capsids and other product related impurities. Empty capsids are separated from vector particles by cesium chloride gradient ultracentrifugation. Using aseptic techniques, cesium chloride is added to the vector ‘Product Intermediate’ with gentle mixing to a final concentration corresponding to a density of 1.35 g/mL. The solution is filtered through a 0.2 μm filter, distributed into ultracentrifugation tubes, and subjected to ultracentrifugation in a Ti50 rotor for approximately 24 h at 15° C. Following centrifugation, the tubes are removed from the rotor, wiped with Septihol, and brought into the BSC. Each tube is clamped in a stand and subjected to focused illumination to assist in visualization of bands. Two major bands are typically observed, the upper band corresponding to empty capsids, and the lower band corresponding to vector particles. The lower band is recovered from each tube with a sterile needle attached to a sterile syringe. Vector recovered from each tube is combined, and samples are taken for in-process bioburden, endotoxin, and vector titer. The pooled material is distributed into sterile 50 mL polypropylene conical tubes labeled ‘Product Intermediate: Post CsCl Gradient’, and stored immediately at −80° C. until the next process step.

8.3.2.6 Buffer Exchange by Tangential Flow Filtration

After testing and release for pooling, batches of vector purified through the CsCl banding process step are combined and subjected to diafiltration by TFF to produce the Bulk Vector. Based on titering of samples obtained from individual batches, the volume of the pooled vectors is adjusted using calculated volume of sterile diafiltration buffer. Depending on the available volume, aliquots of the pooled, concentration adjusted vector are subjected to TFF with single use, TFF devices. Devices are sanitized prior to use and then equilibrated in Diafiltration buffer. Once diafiltration process is complete, the vector is recovered from the TFF apparatus in a sterile bottle. The material is labeled “Pre-0.2 m Filtration Bulk”. The material is further processed immediately.

8.3.2.7 Formulation and 0.2 μm Filtration to Prepare Bulk Vector

Batches prepared by individual TFF units are pooled together and mixed by gentle swirling in a 500 mL sterile bottle. The pooled material is then passed through a 0.22 μm filter to prepare the Bulk Vector. The pooled material is sampled for Bulk Vector and reserved QC testing, and then aliquoted into sterile 50 mL polypropylene tubes, labeled ‘Bulk Vector’, and stored at −80° C. until the next step.

8.4. Testing of Vector

Characterization assays including serotype identity, empty particle content and transgene product identity are performed. Descriptions of all the assays appear below.

8.4.1 Genomic Copy (GC) Titer

An optimized quantitative PCR (oqPCR) assay is used to determine genomic copy titer by comparison with a cognate plasmid standard. The oqPCR assay utilizes sequential digestion with DNase I and Proteinase K, followed by qPCR analysis to measure encapsidated vector genomic copies. DNA detection is accomplished using sequence specific primers targeting the RBG polyA region in combination with a fluorescently tagged probe hybridizing to this same region. Comparison to the plasmid DNA standard curve allows titer determination without the need of any post-PCR sample manipulation. A number of standards, validation samples and controls (for background and DNA contamination) have been introduced into the assay. This assay has been qualified by establishing and defining assay parameters including sensitivity, limit of detection, range of qualification and intra and inter assay precision. An internal AAV8 reference lot was established and used to perform the qualification studies.

8.4.2 Potency Assay

An in vivo potency assay was designed to detect human LDLR vector-mediated reduction of total cholesterol levels in the serum of a double knock-out (DKO) LDLR−/− Apobec−/− mouse model of HoFH. The basis for the development of the in vivo potency assay is described in section 4.3.5.11. To determine the potency of the AAV8.TBG.hLDLR vector, 6-20 week old DKO mice are injected IV (via tail vein) with 5×10¹¹ GC/kg per mouse of the vector diluted in PBS. Animals are bled by retroorbital bleeds and serum total cholesterol levels are evaluated before and after vector administration (day 14 and 30) by Antech GLP. Total cholesterol levels in vector-administered animals are expected to decline by 25%-75% of baseline by day 14 based on previous experience with vector administration at this dose. The 5×10¹¹ GC/kg per mouse dose was chosen for the clinical assay based on the anticipated range of total cholesterol reduction which would allow for the evaluation of changes in vector potency over the course of stability testing.

8.4.3 Vector Capsid Identity: AAV Capsid Mass Spectrometry of VP3

Confirmation of the AAV2/8 serotype of the vector is achieved by an assay based upon analysis of peptides of the VP3 capsid protein by mass spectrometry (MS). The method involves multi-enzyme digestion (trypsin, chymotrypsin and endoproteinase Glu-C) of the VP3 protein band excised from SDS-PAGE gels followed by characterization on a UPLC-MS/MS on a Q-Exactive Orbitrap mass spectrometer to sequence the capsid protein. A tandem mass spectra (MS) method was developed that allows for identification of certain contaminant proteins and deriving peptide sequence from mass spectra.

8.4.4 Empty to Full Particle Ratio

Vector particle profiles using analytical ultracentrifugation (AUC) Sedimentation velocity as measured in an analytical ultracentrifuge are an excellent method for obtaining information about macromolecular structure heterogeneity, difference in confirmation and the state of association or aggregation. Sample was loaded into cells and sedimented at 12000 RPM in a Beckman Coulter Proteomelab XL-I analytical ultracentrifuge. Refractive index scans were recorded every two minutes for 3.3 hours. Data are analyzed by a c(s) model (Sedfit program) and calculated sedimentation coefficients plotted versus normalized c(s) values. A major peak representing the monomeric vector should be observed. The appearance of peaks migrating slower than the major monomeric peak indicate empty/misassembled particles. The sedimentation coefficient of the empty particle peak is established using empty AAV8 particle preparations. Direct quantitation of the major monomeric peak and preceding peaks allow for the determination of the empty to full particle ratio.

8.4.5 Infectious Titer

The infectious unit (IU) assay is used to determine the productive uptake and replication of vector in RC32 cells (rep2 expressing HeLa cells). Briefly, RC32 cell in 96 well plates are co-infected by serial dilutions of vector and a uniform dilution of Ad5 with 12 replicates at each dilution of rAAV. Seventy-two hours after infection the cells are lysed, and qPCR performed to detect rAAV vector amplification over input. An end-point dilution TCID50 calculation (Spearman-Karber) is performed to determine a replicative titer expressed as IU/ml. Since “infectivity” values are dependent on particles coming into contact with cells, receptor binding, internalization, transport to the nucleus and genome replication, they are influenced by assay geometry and the presence of appropriate receptors and post-binding pathways in the cell line used. Receptors and post-binding pathways critical for AAV vector import are usually maintained in immortalized cell lines and thus infectivity assay titers are not an absolute measure of the number of “infectious” particles present. However, the ratio of encapsidated GC to “infectious units” (described as GC/IU ratio) can be used as a measure of product consistency from lot to lot. The variability of this in vitro bioassay is high (30-60% CV) likely due to the low infectivity of AAV8 vectors in vitro.

8.4.6 Transgene Expression Assay

Transgene expression is evaluated in livers harvested from LDLR−/− Apobec−/− mice that receive 1×10¹⁰ GC (5×10¹¹ GC/kg) of the AAV8.TBG.hLDLR vector. Animals dosed 30 days earlier with vector are euthanized, livers harvested and homogenized in RIPA buffer. 25-100 ug of total liver homogenate is electrophoresed on a 4-12% denaturing SDS-PAGE gel and probed using antibodies against human LDLR to determine transgene expression. Animals that receive no vector or an irrelevant vector is used as controls for the assay. Animals treated with vector are expected to show a band migrating anywhere from 90-160 kDa due to post-translational modifications. Relative expression levels are determined by quantifying the integrated intensity of the bands.

(Sequence Listing Free Text)
The following information is provided for sequences containing free text under
numeric identifier <223>.

| SEQ ID NO: | |
|---|---|
| (containing | |
| free text) | Free text under <223> |
| 1 | <221> misc_feature |
| | <222> (1)..(254) |
| | <223> exon |
| | <220> |
| | <221> misc_feature |
| | <222> (188)..(2770) |
| | <223> LDLR isoform 1 encoded by full-length CDS, 188-2770; |
| | other |
| | variants encoded by alternative splice variants missing an |
| | exon; |
| | most common variant missing fourth exon or twelfth exon |
| | <220> |
| | <221> misc_signal |
| | <222> (188)..(250) |
| | <220> |
| | <221> misc_feature |
| | <222> (251)..(2767) |
| | <223> Mature protein of isoform 1 |
| | <220> |
| | <221> misc_feature |
| | <222> (255)..(377) |
| | <223> exon |
| | <220> |
| | <221> misc_feature |
| | <222> (378)..(500) |
| | <223> exon |
| | <220> |
| | <221> misc_feature |
| | <222> (501)..(881) |
| | <223> exon |
| | <220> |
| | <221> misc_feature |
| | <222> (882)..(1004) |
| | <223> exon |
| | <220> |
| | <221> misc_feature |
| | <222> (1005)..(1127) |
| | <223> exon |
| | <220> |
| | <221> misc_feature |
| | <222> (1128)..(1247) |
| | <223> exon |
| | <220> |
| | <221> misc_feature |
| | <222> (1248)..(1373) |
| | <223> exon |
| | <220> |
| | <221> misc_feature |
| | <222> (1374)..(1545) |
| | <223> exon |
| | <220> |
| | <221> misc_feature |
| | <222> (1546)..(1773) |
| | <223> exon |
| | <220> |
| | <221> misc_feature |
| | <222> (1774)..(1892) |
| | <223> exon |
| | <220> |
| | <221> misc_feature |
| | <222> (1893)..(2032) |
| | <223> exon |
| | <220> |
| | <221> misc_feature |
| | <222> (2033)..(2174) |
| | <223> exon |
| | <220> |
| | <221> misc_feature |
| | <222> (2175)..(2327) |
| | <223> exon |
| | <220> |
| | <221> misc_feature |
| | <222> (2328)..(2498) |
| | <223> exon |
| | <220> |
| | <221> polyA_signal |
| | <222> (5252)..(5257) |
| | <220> |
| | <221> polyA_site |
| | <222> (5284)..(5284) |
| 4 | <223> Artificial hLDLR |
| | <220> |
| | <221> misc_feature |
| | <222> (1)..(2583) |
| | <223> Artificial hLDLR coding sequence |
| 5 | <223> Adeno-associated virus 8 vp1 capsid protein |
| 6 | <223> pAAV.TBG.PI.hLDLRco.RGB |
| | <220> |
| | <221> repeat_region |
| | <222> (1)..(130) |
| | <223> 5′ ITR |
| | <220> |
| | <221> enhancer |
| | <222> (221)..(320) |
| | <223> Alpha mic/bik |
| | <220> |
| | <221> enhancer |
| | <222> (327)..(426) |
| | <223> Alpha mic/bik |
| | <220> |
| | <221> promoter |
| | <222> (442)..(901) |
| | <223> TBG |
| | <220> |
| | <221> TATA signal |
| | <222> (885)..(888) |
| | <223> TATA |
| | <220> |
| | <221> CDS |
| | <222> (969)..(3551) |
| | <223> codon optimized hLDLR |
| | <220> |
| | <221> polyA_signal |
| | <222> (3603)..(3729) |
| | <223> Rabbit globin poly A |
| | <220> |
| | <221> repeat_region |
| | <222> (3818)..(3947) |
| | <223> 3′ ITR |
| | <220> |
| | <221> rep_origin |
| | <222> (4124)..(4579) |
| | <223> fl ori |
| | <220> |
| | <221> misc_feature |
| | <222> (4710)..(5567) |
| | <223> AP(R) |
| | <220> |
| | <221> rep_origin |
| | <222> (5741)..(6329) |
| | <223> Origin of replication |
| 7 | <223> Synthetic Construct |

All publications cited in this specification are incorporated herein by reference in their entirety, as is U.S. Provisional Patent Application No. 62/782,627, filed Dec. 20, 2018. Similarly, the SEQ ID NOs which are referenced herein and which appear in the appended Sequence Listing labeled “16-7717C2PCT_20191210_SequenceListing_ST25”, dated Dec. 10, 2019 and is 64,936 bytes in size. are incorporated by reference. While the invention has been described with reference to particular embodiments, it will be appreciated that modifications can be made without departing from the spirit of the invention. Such modifications are intended to fall within the scope of the appended claims.

1. A regimen for use in the treatment of familial hypercholesterolemia (FH), comprising administering to a patient a suspension comprising a recombinant adeno-associated virus (rAAV) viral particle which comprises a vector genome packaged in an AAV8 capsid, wherein the vector genome comprises AAV inverted terminal repeats (ITRs) and a nucleic acid sequence encoding a human low-density lipoprotein (LDL) receptor (hLDLR) operably linked to a liver specific promoter, wherein the suspension is administered to the patient at a dose of about 2.5×10¹³ Genome Copy (GC) of the rAAV viral particle per kilogram (kg) body weight of the patient, and wherein the genome copy is determined by optimized quantitative polymerase chain reaction (oqPCR) or digital droplet polymerase chain reaction (ddPCR).

2-26. (canceled)

27. A suspension for use in the treatment of familial hypercholesterolemia (FH), comprising a recombinant adeno-associated virus (rAAV) viral particle which comprises a vector genome packaged in an AAV8 capsid, wherein the vector genome comprises AAV inverted terminal repeats (ITRs) and a nucleic acid sequence encoding a human low-density lipoprotein (LDL) receptor (hLDLR) operably linked to a liver specific promoter, wherein the suspension is suitable for administering to a patient at a dose of about 2.5×10¹³ Genome Copy (GC) of the rAAV viral particle per kilogram (kg) body weight of the patient, and wherein the genome copy is determined by optimized quantitative polymerase chain reaction (oqPCR) or digital droplet polymerase chain reaction (ddPCR).

28-30. (canceled)

31. A method for treating a patient having familial hypercholesterolemia (FH), comprising administering to the patient a suspension comprising a recombinant adeno-associated virus (rAAV) viral particle, wherein the rAAV viral particle comprises a vector genome packaged in an AAV8 capsid, wherein the vector genome comprises AAV inverted terminal repeats (ITRs) and a nucleic acid sequence encoding a human low-density lipoprotein (LDL) receptor (hLDLR) operably linked to a liver specific promoter, wherein the patient is administered with about 2.5×10¹³ Genome Copy (GC) of the rAAV viral particle per kilogram (kg) body weight of the patient, and wherein the genome copy number or titer is determined by optimized quantitative polymerase chain reaction (oqPCR) or digital droplet polymerase chain reaction (ddPCR).

32. The method according to claim 31, wherein the suspension is an aqueous solution comprising the rAAV viral particle and a formulation buffer.

33. The method according to claim 32, wherein the formulation buffer comprises a phosphate buffered saline and a surfactant.

34. The method according to claim 31, wherein the suspension having a rAAV Genome Copy (GC) titer of at least 1×10¹³ GC/ml.

35-37. (canceled)

38. The method according to claim 31, further comprising administering the patient with a steroid.

39. The method according to claim 31, wherein the suspension comprises the rAAV viral particle at a concentration or titer of at least 1×10¹³ GC/ml.

40. The method according to claim 31, wherein the liver specific promoter is a thyroxine-binding globulin (TBG) promoter.

41. The method according to claim 40, wherein the TBG promoter is a human TBG promoter.

42. The method according to claim 31, wherein hLDLR nucleic acid sequence comprises a sequence of SEQ ID NO: 2 or 4, or nucleotide (nt) 969 to nt 3551 of SEQ ID NO: 6.

43. The method according to claim 31, wherein the vector genome further comprises one or more of an intron, an enhancer and a polyA signal.

44. The method according to claim 31, wherein the enhancer is an alpha 1 microglobulin/bikunin enhancer (alpha mic/bik) enhancer.

45. The method according to claim 31, wherein the polyA signal is a rabbit beta globin polyA.

46. The method according to claim 31, wherein the vector genome comprises a sequence of nucleotide (nt) 1 to nt 3947 of SEQ ID NO: 6.

47. The method according to claim 31, wherein the suspension or the formulation buffer further comprises a surfactant.

48. The method according to claim 47, wherein the surfactant is a Poloxamer.

49. The method according to claim 47, wherein the surfactant is present in a concentration of about 0.0005% to about 0.001% of the composition.

50. The method according to claim 31, wherein the suspension has a pH ranging 6.5 to 8 or 7 to 7.5.

51. The method according to claim 31, wherein the suspension further comprises a formulation buffer which comprises 180 mM NaCl, 10 mM Na phosphate, and 0.001% Poloxamer 188, at pH 7.3.

52. The method according to claim 31, further comprising administering the suspension to the patient via a peripheral vein infusion.

53. The method according to claim 31, wherein the patient is a human patient diagnosed with Homozygous FH (HoFH).

54. The method according to claim 31, wherein the patient is co-treated with an immunosuppressant.

55. The method according to claim 54, wherein the patient is treated with a steroid co-therapy for about 14 weeks.

56. The method according to claim 54, wherein the immunosuppressant is administered to the patient at a tapering dose equivalent to an initial dose of about 40 mg/day prednisone for one day before the administration of the rAAV suspension (i.e., day −1) to about week 8 post-dosing with the rAAV.

57. The method according to claim 56, wherein the patient is further administered with the immunosuppressant by a 10 mg dose decrease/week for each of weeks 9 and 10, and a 5 mg dose decrease/week for each of weeks 11, 12 and 13, such that the immunosuppressant is discontinued after week 14.

58. The method according to claim 54, wherein the immunosuppressant is the steroid prednisone.
